(19) United States
(12) Patent Application Publication
(10) Pub. No.: US 2020/0101014 A1
(43) Pub. Date: Apr. 2, 2020

(54) Stable Theranostic and Therapeutic Nanoemulsions Using Triphilic Semifluorinated Amphiphiles

(71) Applicant: Wisconsin Alumni Research Foundation, Madison, WI (US)

(72) Inventors: Sandro MECOZZI, Madison, WI (US); Alexa BARRES, Madison, WI (US)

(73) Assignee: Wisconsin Alumni Research Foundation, Madison, WI (US)

(21) Appl. No.: 16/494,719

(22) PCT Filed: Apr. 2, 2018
(86) PCT No.: PCT/US2018/025741

§ 371 Date: Sep. 16, 2019

Related U.S. Application Data
(60) Provisional application No. 62/480,996, filed on Apr. 3, 2017.

(51) Int. Cl. A61K 9/107; A61K 31/337; A61K 49/18; A61K 47/02; A61K 9/00; A61K 47/34

(52) U.S. Cl. CPC A61K 9/1075; A61K 31/337; A61K 47/34; A61K 47/02; A61K 9/0019; A61K 49/1806

(57) ABSTRACT

The present invention provides therapeutic formulations, including therapeutic nanoemulsions, and related methods for the in vivo delivery of hydrophobic compounds. Formulations and methods of the invention include semifluorinated block copolymers and an imaging compound to form a theranostic nanoemulsion, capable of forming a stable nanoemulsion. In certain embodiments, emulsion-based formulations are provided that are capable of formulating, delivering and releasing amounts of hydrophobic drugs effective for a range of clinical applications, including treating cancer and fungal infections in patients. In certain embodiments, emulsion-based formulations are provided that are capable of supporting controlled release, for example, over a range of rates useful for clinical applications including sustained release.

### RESEARCH OR DEVELOPMENT

This invention was made with United States government support awarded by the following agencies: National Institutes of Health (Grant No. R21EB021431) and National Science Foundation Graduate Research Fellowship Program (Grant No. DGE-1747503). The United States has certain rights in this invention.

### BACKGROUND

Administration of hydrophobic drugs presents practical challenges due to the limited water solubility of this class of pharmaceuticals. Accordingly, a primary focus of drug delivery research is development of effective approaches for the formulation and controlled delivery for this class of important pharmaceutical agents. Nanoemulsions are a particularly promising delivery vehicle for these applications given their intrinsic stability and potential to access useful pharmacokinetic properties for drug administration, absorption and targeting.

Nanoemulsions are composed of nanoscale droplets of one immiscible liquid dispersed within another. In the context of many pharmaceutical applications, for example, the dispersed droplet phase of a nanoemulsion provides a central oil core, stably dispersed in an aqueous phase that can act as an effective reservoir for hydrophobic drugs. Nanoemulsions for delivery applications often incorporate one or more surfactants and/or stabilizers to facilitate stabilization and improve drug solubilization of the dispersed phase. As nonequilibrium systems, preparation of a nanoemulsion typically involves an input of energy, for example, using a microfluidizer, high pressure homogenizer or ultrasonicator.

Typical droplet sizes for nanoemulsions for the delivery of pharmaceuticals are in the range of about 20-500 nm. The small droplet size characteristic of nanoemulsions provides benefits supporting their use as vehicles for pharmaceutical delivery. First, the small droplet size and lower surface tension between dispersed and aqueous phases decrease the rates of droplet agglomeration and precipitation processes so as to substantially limit the potential for phase separation via sedimentation, flocculation, coalescence and creaming. As a result, nanoemulsions are typically more kinetically stable than other types of emulsions. Second, the nanosized dimensions of the droplets allow for effective in vivo administration, for example via drug absorption from the gastrointestinal tract, intravenously, or penetration of the skin barrier. Third, the large interfacial area provided by the small size of the dispersed droplets allows for the potential to effectively control drug release over a clinically useful range. Accordingly, nanoemulsions have significant potential for providing rapid, sustained or targeted delivery and release of hydrophobic drugs.

A large variety of diverse nanoparticles have been reported in the literature in various medical applications. However, independently from the composition and sophistication of the particles, nanocarriers to be used in drug delivery must obey at least an elementary set of fundamental rules. They must be safe to use in humans. In case the particles are injected intravenously, it is important that they will not bioaccumulate in specific organs, but will eventually be degraded and/or excreted within a reasonable time. The nanoparticles must be able to bind and encapsulate amounts of drugs consistent with the proposed medical use. The drug/nanoparticle complex must be stable in physiological conditions, for instance in blood, for the time needed to exert a biological action.

Often, these requirements are inconsistent with each other. For instance, a nanoparticle very stable in blood may bioaccumulate because it is sequestered in the liver. This is the fate of several dendrimer-based particles and other covalently linked nanoparticles. In other instances, nanoparticles composed of monomers that aggregate in aqueous solution to form specific systems such as micelles or vesicles, may be devoid of toxicity because the monomers are of a size that can eventually be filtered through the kidneys. However, the same process of hydrophobic recognition that makes possible a non-covalent aggregate, leads, in the majority of cases, to low stability of the aggregates in physiological conditions, especially in blood. As a consequence, the encapsulated drugs are released shortly after the nanoparticles are injected in circulation.

A recent, necessarily partial, compilation of reviews on nanotechnologies applied to medicine, used four volumes to describe the most known applications. Among the various nanoparticles that have been used in biomedical research, nanoemulsions (NEs) occupy a special place. NEs can be defined as oil-in-water (O/W) or water-in-oil (W/O) emulsions that consist of droplets having an average size below 500 nm, and most often between 50 and 250 nm. Nanoemulsions have been used as mucosal vaccines, topical antimicrobials, contrast agents, and, most important, as drug nanocarriers. NE-based drug delivery applications are found in ocular, parenteral, oral, dermal, nasal delivery. NEs have a number of characteristics that make them highly suitable for the delivery of hydrophobic drugs:

A large amount of drug can be efficiently encapsulated in NEs. For instance, a standard drug therapeutic concentration of 1 mg/ml can be achieved by using other noncovalent aggregates such as micelles. When solid hydrophobic drugs need to be dissolved in an emulsion, they are first solubilized in an oil phase composed, for example, of 2-octyl-1-dodecanol (FDA approved) or pharmaceutical grade soybean oil. Saturated solutions of the drug to be emulsified can be used as the oil phase. Addition of a suitable surfactant and subsequent microfluidization can then lead to a nanoemulsion containing relatively large amounts of drug.

The size of the nanoparticles in a nanoemulsion can be modulated simply by choosing the air pressure in the microfluidizer and the number of cycles to be used. By varying these two parameters one can make NEs composed of nanoparticles in the size range of 50-300 nm. Specific pharmacokinetics requirements for the drug to be delivered can dictate a specific size of the nanoparticles to be used. This extreme flexibility in the size of the nanoparticles is a peculiar property of the way nanoemulsions are fabricated. Interestingly, different particle sizes can also lead to different nanoemulsion stability, something to be taken into account during the preparation of a NE.

NEs also share one additional, although negative, property of all drug delivery systems based on self-assembling nanoparticles, namely, poor blood circulation times. This is due to the fact that all non-covalent assemblies made in aqueous solution are based on amphiphilic molecules that use the hydrophobic effect and interaction both to assemble in nanoparticles and also to encapsulate hydrophobic drugs. These complexes are always in dynamic equilibrium with their monomers. At all times, a number of unimers (monomers composing the aggregate) are released in solution and a similar number are brought back to the aggregate. This is a standard process related to the behavior of self-assembling amphiphiles in water. However, this process becomes a reason of concern when the same aggregates are put in blood, for instance, during intravenous delivery. When unimers are released in blood they almost immediately interact with hydrophobic molecules present in blood. Albumin is a protein that has a number of hydrophobic patches on its surface. Unimers containing a hydrophobic domain (a requirement for making an amphiphile) will be bound by albumin and subtracted to the equilibrium with the self-assembled aggregate. This process leads to the very quick dissociation of the aggregates in physiological conditions. For instance, most micelles have a half-life in blood between a few minutes and several hours, depending on the structure of the amphiphile. The same micelles will be highly stable in a simple aqueous solution not containing any hydrophobic component. Of course, a similar problem is also present with NEs. The stability of a particle in a nanoemulsion is related to the surfactant forming a shield around a particle of the oil phase. The effectiveness of this shield is related to its compactness, that is, to the number of surfactant molecules that surround each oil particle in an aqueous solution. If the molecules of surfactant are removed because of interactions between the surfactant unimers and the blood hydrophobic components, the emulsion particles will eventually be destabilized and the oil phase will be released, leading to immediate systemic delivery of all the drug that had been injected with the emulsion.

Theranostics is a term describing dual therapeutic and diagnostic character. Theranostic nanomedicine is an expanding field geared towards personalized medicine that combines simultaneous treatment with diagnostics to better serve the patient. This field frequently involves administration of a chemotherapy, gene therapy, or radiation therapy with an imaging agent. The administration of nuclear magnetic resonance imaging (MRI) continues to expand due to its noninvasive nature, deep tissue penetration, excellent sensitivity, and widespread application. Most frequently, ¹H nuclei are utilized due to their high sensitivity of 1.0, natural abundance of 99%, and the large amount of water present in anatomical systems. Though less common, ¹⁹F nuclei can be used easily for MRI with similar sensitivity to proton, 0.83, and an even higher natural abundance of 100%. In order to clearly visualize tissues in traditional ¹H MRI, a contrast agent is commonly required; two of the most common include chelated gadolinium and superparamagnetic iron oxide (SPIO). Both metallic compounds are very efficient at indirect imaging but have exhibited several toxicity concerns including oxidative stress, altered gene expression, and in extreme cases, kidney failure. In a similar manner, fluorocarbons with high signal intensity can be used to image anatomical systems.

It will be appreciated from the foregoing that emulsion-based delivery systems for the formulation and administration of hydrophobic drugs are needed. Systems and formulations are needed that are capable of providing stable formulation of hydrophobic drugs exhibiting sparing solubility in aqueous solutions, particularly, in concentrations supporting a range of important clinical applications. Systems and formulations are needed exhibiting a high degree of biocompatibility, low toxicity and pharmacokinetic properties supporting controlled delivery and targeting of hydrophobic drugs. Additionally, systems and formulations that have theranostic capacity are also desired. Thus, there remains a need for developing physiologically stable and safe drug delivery systems and theranostics that can be used for the aqueous solubilization and effective delivery of consistent amounts of hydrophobic drugs, for the treatment of solid tumors as well as for the sustained delivery of antifungals and antibiotics.

### SUMMARY OF THE INVENTION

The present invention provides therapeutic formulations, including therapeutic nanoemulsions, and related methods for the in vivo delivery of hydrophobic compounds comprised within an oil, such as in an oil-in-water emulsion, together with, in some embodiments, an MRI-active perfluorinated imaging agent to form a theranostic nanoemulsion. Formulations and methods of the invention include semifluorinated block copolymers as described herein. In certain embodiments, emulsion-based formulations are provided that are capable of formulating, delivering and releasing amounts of hydrophobic drugs effective for a range of clinical applications, including anti-fungal and anti-cancer applications. In certain embodiments, emulsion-based formulations according to the invention are provided that are capable of supporting controlled release and extended release, for example, over a range of rates useful for clinical applications including extended and sustained release.

In certain embodiments, nanoemulsion formulations of the present invention include linear and branched semifluorinated block copolymers and MRI-active fluorous compounds having compositions resulting in enhanced stability with respect to droplet size by decreasing the rate of Ostwald ripening, coagulation and/or phase separation processes. Therapeutic formulations of the present invention also provide a high degree of versatility, as the amount and composition of the semi-fluorinated block copolymer component (e.g., length and composition of the hydrophilic block, length and composition of the fluorophilic block, length and composition of the hydrophobic block, etc.) and the amount and chemical composition of the MRI-active fluorous compounds may be selectively adjusted to: (i) enhance stability under delivery conditions, (ii) optimize the kinetics of release of a hydrophobic compound for a specific application (e.g. provide slower release rates), (iii) provide direct imaging of the location of the emulsions in the body and (iv) enhance the overall formulation stability of therapeutic nanoemulsions under storage conditions (e.g., increase useful shelf life).

In some embodiments of the formulations of the present invention, the nanoemulsions are capable of being imaged directly. By incorporating an MRI-active perfluorinated compound such as, for example, perfluoro-15-crown-5-ether (PFCE) into a nanoemulsion of the invention, the present invention takes advantage of the intrinsic properties of ¹⁹F nuclei in order to form theranostic nanoemulsions. Perfluoro-15-crown-5-ether (PFCE), for example, has high signal intensity and low toxicity compared to other MRI-active perfluorinated compounds. Due to the low abundance of fluorine in biological systems, excluding bone and teeth, there will be an excellent signal-to-noise ratio as well as a near-zero background.

In an embodiment, the present formulations comprise linear semi-fluorinated block copolymers having hydrophilic, hydrophobic and fluorophilic blocks, and optionally an MRI-active imaging agent, such as a perhalogenated fluorous compound, which are capable of stabilizing an emulsion of a clinically effective amount of a hydrophobic compound, such as an anti-cancer or anti-fungal agent in an oil-based phase, dispersed in an aqueous solution. Where the MRI-active imaging agent is incorporated into the nanoemulsion, the nanoemulsion is also capable of being detected in an MRI system.

In the present invention, the term “amphiphile” is used to designate a molecule with two or more chemically different domains on the same molecule, e.g., like hydrophobic and hydrophilic. In the polymers of the invention, which are referred to as “amphiphile” or “amphiphiles,” there are in fact three domains, a fluorophilic domain, a hydrophobic domain, and a hydrophilic domain. The amphiphilic polymers of the invention may be referred to by the term “triphilic” to refer to the fact that the amphiphiles of the invention have three domains, not two domains.

Amphiphilic polymers, such as the semifluorinated block copolymers of the invention, can form nanoscale assemblies including the inventive nanoemulsions of the present invention, in aqueous solution, and have vast potential in drug delivery. The tunability of the polymer design results in varied nanoparticle sizes and shapes, as desired by the particular application. Incorporation of a fluorous block into the amphiphile provides an enhanced driving force towards assembly and particle stability overall, in comparison to typical hydrocarbon polymers. This is a result of the unique properties of fluorous moieties in solution including dual hydrophobicity and lipophobicity, high thermal and chemical stability, high surface tension, and biological inertness. In addition, due to the extremely strong carbon-fluorine bond (472 kJ/mol) these semifluorinated polymers have very low polarizability and rigid, helical structures. All of these properties result in the formation of an energetically favored fluorous phase in solution and with it, increased emulsion stability.

Colloidal nanoemulsions are kinetically stable nanoparticles in the size range of 100-400 nm. These particles are formulated by the input of a high amount of energy into a solution of two immiscible liquids and an appropriate surfactant. In the nanoemulsions of the invention, oil-in-water nanoemulsions are the focal points which result in oil droplets stabilized by the semifluorinated block copolymers of the invention, surrounded by an aqueous environment, such as saline. Nanoemulsions of this design are especially advantageous over other nanoparticle systems due to their large oil droplet core, perfect for the solubilization of hydrophobic drugs such as chemotherapeutics and antifungal agents. This nanoscale system is able to exploit the enhanced permeability and retention (EPR) effect to preferentially accumulate within tumor tissue over healthy tissue due to leaky tumor vasculature, a poorly functioning tumor lymphatic system, and the size of the nanoemulsion particles. This type of selective delivery to tumor tissue as well as the high drug payload afforded by nanoemulsions makes them a potential potent therapy for use in cancer treatment.

The present invention, in one embodiment includes a new semifluorinated, triblock ABC copolymer comprising hydrophilic, fluorophilic, and lipophilic segments that can form triphilic nanoemulsions, according to the invention, as described herein. This innovative triphilic design is ideal for solid chemotherapeutic drug delivery as well as use as a theranostic system by the addition of an imaging agent. The introduction of the fluorocarbon moiety here has allowed for the development of nanoparticles that can modulate drug retention and exhibit enhanced kinetic stability.

In one embodiment, the copolymer designated herein as M2F8H18, uniquely demonstrates that hydrophobic drugs can be encapsulated with high loading and retention within the large oil droplet core. The lipophilic block of the polymer penetrates the oil core, stabilizing the emulsions and interacting non-covalently with the drug and oil. Drug release from the core is modulated by the fluorous shell of the polymer. Particle solubility, packing, and “stealth” from blood proteins is accomplished by the hydrophilic block on the corona. The addition of a perfluorocarbon excipient provides further emulsion stability, enhanced drug retention, as well as potent ¹⁹F-magnetic resonance imaging (MRI) signal and imaging possibilities.

The surface of a nanoemulsion (NEs) of the present invention can be functionalized with targeting agents similarly to how surfactants in micelles and liposomes can also be functionalized. Thus, NEs of the present invention are capable of being used in an active targeting therapy.

The imaging agent can be a perfluorinated moiety. Fluorocarbons are very unusual molecules. Due to the peculiar electronic distribution found on the surface of a fluorocarbon, these molecules are both hydrophobic and lipophobic. This means that when a fluorocarbon such as perfluorohexane is mixed with a hydrophobic molecule such as hexane, and with water, it will generate three different phases as the fluorous molecules will not mix with either hydrophobic molecules or water. The third phase in which the fluorocarbons self-segregate is known as the fluorous phase and the phenomenon that leads to the formation of a fluorous phase is the fluorophobic effect in analogy of the hydrophobic effect. The formation of a fluorous phase can be exploited in the generation of highly stable self-assembled complexes. Furthermore, the fact that a fluorocarbon is both hydrophobic and lipophobic implies that an amphiphilic molecule containing a fluorophilic domain will partially repel binding from blood hydrophobic components. This means that a fluorous amphiphile will self-assemble in aggregates that will be somewhat resistant to blood-induced dissociation.

Thus, therapeutic formulations of the present invention include nanoemulsions including a hydrophobic compound comprised within submicron droplets of an oil-based dispersion, which is dispersed in a continuous phase comprising an aqueous solution, such as a solution isotonic to blood. Thus, in some embodiments, the present invention includes an emulsion for delivery of a therapeutic agent and/or an imaging agent, and said emulsion can include a dispersed phase and a continuous phase. The dispersed phase can comprise oil droplets comprising a hydrophobic liquid, and optionally, a therapeutic agent. The dispersed phase may also comprise semi-fluorinated block copolymers; wherein each of said semi-fluorinated block copolymers independently comprises a hydrophilic block, a hydrophobic block and a fluorophilic block; wherein said fluorophilic block of each of said semi-fluorinated block copolymers is provided between said hydrophobic block and said hydrophilic block. The fluorophilic phase of the polymer may optionally comprise a fluorous imaging agent as described hereinbelow. The continuous phase is an aqueous solution. The structure of the emulsions may be envisioned as shown in FIG. 3, where the dispersed phase is a droplet comprising an inner shell comprising a hydrophobic liquid with the therapeutic agent, preferably a hydrophobic compound, is disposed within the hydrophobic liquid; a mid-layer comprising a fluorophilic shell comprising the fluorophilic block of the copolymer, with the optional fluorous imaging agent disposed within the fluorophilic shell; and an outer layer, also referred to as a hydrophilic corona, comprising the hydrophilic block of the copolymer. The droplet is dispersed within an aqueous solution.

In an aspect, the present invention provides emulsion-based formulations, such as nanoemulsions. For example, in one embodiment, an emulsion of the invention is useful for delivery of a therapeutic agent comprising a hydrophobic drug. Emulsions of this aspect are beneficial, for example, for delivering therapeutic agents comprising a hydrophobic compound to a patient or subject, such as a therapeutic agent which is insoluble or only sparingly soluble in aqueous solution. For example, in some embodiments, hydrophobic therapeutic agents which are soluble in aqueous solutions at concentrations or dosages less than a useful therapeutic amount benefit from the emulsions of the invention, which provide for the ability to deliver a therapeutic or effective amount of the therapeutic agent to a patient or subject.

In an aspect, an emulsion of the invention includes a hydrophobic liquid in which the hydrophobic therapeutic agent is solubilized and/or dispersed. Systems for solubilizing solid hydrophobic drugs include solubilization in an oil phase as known in the art. Saturated solutions of the drug to be emulsified can be used as the hydrophobic and/or dispersed and/or oil phase. The hydrophobic liquid useful for the invention, in one embodiment, can include any FDA-approved oil, for example, 2-octyl-1-dodecanol (FDA approved) or pharmaceutical grade soybean oil. In one embodiment of the present invention, the hydrophobic liquid includes glycerides, which can include monoglycerides, diglycerides, or triglycerides comprising short, medium or long chain acyl groups. In one embodiment, the triglyceride is a medium chain triglyceride (“MCT”), or mixtures thereof, as known in the art. In one embodiment, a MCT in accordance with the invention includes the following Formula I.

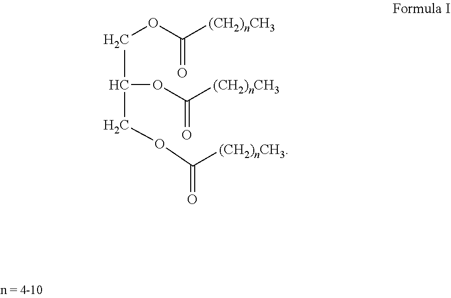

Subsequent microfluidization can then lead to a nanoemulsion containing relatively large amounts of drug.

In an embodiment of this aspect, the emulsion is a nanoemulsion, for example, characterized by a dispersed phase comprising droplets as described elsewhere herein having cross sectional dimensions selected from the range of 20 nm to 1 micron, and optionally selected from the range of 100 nm to 1 micron, or 200 nm to 500 nm, or, alternatively, less than 500 nm, less than 400 nm, less than 300 nm, less than 200 nm, less than 100 nm. The exact size of the droplets may be “tuned” in accordance with the desired use or to maximize stability and/or delivery, as appropriate. In an embodiment of the emulsion, the dispersed phase droplets form a hydrophobic core which comprises a hydrophobic therapeutic agent, and further comprise the hydrophobic block of the semifluorinated block copolymers of the invention. In one embodiment, the emulsion has a hydrophilic exterior shell comprising the hydrophilic blocks of the semi-fluorinated block copolymers of the invention. In one embodiment, the emulsion has a fluorophilic intermediate shell comprising the fluorophilic block of the semi-fluorinated block copolymers. Optionally, the therapeutic formulation of this aspect of the present invention is capable of delivery to a patient via parenteral administration, such as via intravenous injection.

In embodiments, the composition and relative amounts of the components of the emulsions are selected so as to achieve a desired solubilization amount of one or more hydrophobic therapeutic agents, such as a clinically or therapeutically effective amount. In embodiments, the composition and relative amounts of the components of the emulsions are selected so as to achieve a desired or controlled release rate, release timing or targeted delivery of one or more hydrophobic therapeutic agents.

In embodiments, emulsions of the invention comprise semi-fluorinated block copolymers. The structure, composition, size or concentration of the semi-fluorinated block copolymers and polymer block components thereof are optionally selected so as to provide certain properties to the emulsion, such as physical properties, emulsion ripening rate, emulsion stability, therapeutic agent solubility, composition, toxicity, biocompatibility, therapeutic effectiveness, therapeutic agent delivery rate or release rate, immune or other physiological response or any combination of these. In an embodiment, the hydrophilic block and fluorophilic block as each independently polymer terminating blocks, for example, wherein the hydrophobic block is an intermediate block provided directly or indirectly in between the hydrophilic block and fluorophilic block.

In embodiments, for example, the semi-fluorinated block copolymers have a concentration selected from the range of 2 mg mL⁻¹ to 40 mg mL⁻¹, optionally for some applications selected from the range of 5 mg mL⁻¹ to 30 mg mL⁻¹. In some embodiments, the semi-fluorinated block copolymers have a concentration selected from the range of 10 to 20 mg mL⁻¹. In embodiments, the semi-fluorinated block copolymers may have a concentration of between 5 and 30 mM, between 10 and 20 mM. In embodiments, for example, each of the semi-fluorinated block copolymers independently has a molecular weight selected from the range 1000 Da to 20,000 Da, and optionally for some applications 1100 Da to 14,000 Da, 2000 Da to 12,000 Da, or 4000 Da to 8,000 Da.

In certain embodiments, the structure, composition or size of the hydrophilic block of the semi-fluorinated block copolymers are selected so as to make stable emulsion-based formulations from a wide range of hydrophobic therapeutic agents. In exemplary embodiments, the hydrophilic block of each of the semi-fluorinated block copolymers is a polymer terminating group.

In embodiments, the hydrophilic block is selected from the group consisting of a polyoxygenated polymer block, a poly(vinylpyrrolidone) block, a poly(acrylic) block, a polyacrylamide block, a polyoxazoline block, a polysaccharide block and a chitosan derivative block. In an embodiment, the hydrophilic block is a polyoxygenated block, such as a poly(ethylene glycol) block. In exemplary embodiments, the hydrophilic block is a poly(ethylene glycol) block, for example, having a molecular weight selected from the range of 1000 g mol⁻¹ to 40,000 g mol⁻¹, optionally for some applications selected from the range of 1000 g mol⁻¹ to 20,000 g mol⁻¹, optionally for some applications selected from the range of 1000 g mol⁻¹ to 15,000 g mol⁻¹, and optionally for some applications selected from the range of 1000 g mol⁻¹ to 10,000 g mol⁻¹. In some embodiments, selection of the size/molecular weight of the poly(ethylene glycol) block establishes the release rate of a hydrophobic therapeutic agent and/or stability of the nanoemulsion with respect to ripening, coagulation and phase separation processes. In some embodiments, the hydrophilic block is directly linked to the fluorophilic block.

In certain embodiments, the structure, composition or size of the fluorophilic block of the semi-fluorinated block copolymers are selected so as to assist in making stable emulsion-based formulations from a wide range of hydrophobic therapeutic agents. In exemplary embodiments, the fluorophilic block is a perfluorinated carbon moiety having between 3 and 16 carbons, between 6 and 12 carbons, or between 6 and 10 carbons, or 8 carbons. Embodiments may include fluorophilic blocks having between 6 and 32 carbon-fluorine bonds, or between 8 and 24 carbon-fluorine bonds, or between 12 and 20 carbon-fluorine bonds, or about 16 carbon-fluorine bonds. Optionally for some embodiments, the fluorocarbon moiety is a perfluorinated moiety. In some embodiments, each fluorophilic block is independently a fluorinated alkyl group having a length greater than or equal to 3 carbons and optionally for some applications greater than or equal to 6 carbons, and optionally for some embodiments greater than or equal to 10 carbons. In some embodiments, the fluorophilic block is a fluorinated alkyl group having a length of 3 to 20 carbons, optionally for some applications of 3 to 15 carbons and optionally for some applications 6 to 15 carbons. In some embodiments, for example, each fluorophilic block is independently a perfluorinated alkyl group having a length of 3 to 15 carbons, and optionally 3 to 8 carbons. In exemplary embodiments, the fluorophilic block is directly linked to both the hydrophilic block and the hydrophobic block.

In some embodiments, the fluorophilic block is linked to the hydrophilic block and independently to the hydrophobic block via a linking moiety selected from the group consisting of a substituted or unsubstituted group selected from an ether group, a carbamate group, an amide group, a carboxyl group, an ester group, an alkyl group, an alkylene group, an amino group or any combination of these. These groups may optionally contain heteroatoms. The linker may comprise null, —O—, —O (CH₂)ₑ—, —(CH₂)ₑO—, —(CH₂)ₑ—, —(CH₂)ₑO(CH₂)_{f}—, —(CH₂)ₑNR¹(CH₂)_{f}—, —(CH₂)ₑOCNR²(CH₂)_{f}—, —(CH₂)ₑCONR³(CH₂)_{f}—, —(CH₂)ₑNR⁴COO(CH₂)_{f}—, —(CH₂)ₑNR⁵CO(CH₂)_{f}— or —(CH₂)ₑNR⁶CONR⁷(CH₂)_{f}—; where each of R¹-R⁷ is independently hydrogen, methyl, or C₁-C₅ alkyl; and where each of e and f is independently an integer selected from the range of 0 to 5; and where m is 0 or 1 and n is 0 or 1. In some embodiments, the linking moiety is —(CH₂)ₑO(CH₂)_{f}—, where e and f are each independently 1.

In certain embodiments, the structure, composition or size of the hydrophobic block of the semi-fluorinated block copolymers are selected so as to make stable emulsion-based formulations from a wide range of hydrophobic therapeutic agents. In embodiments, for example, the hydrophobic block is selected from the group consisting of one or more linear or branched C₅-C₂₀ alkyl block, a poly (E-caprolactone) block, a poly(lactic acid) block; a poly(propylene glycol) block; a poly(amino acid) block; a poly(ester) block and poly(lactic-co-glycolic acid) block. In some embodiments, the hydrophobic block is as substituted or unsubstituted C₅-C₂₀ alkyl group having one or more unsaturations, optionally a substituted or unsubstituted C₅-C₁₀ alkyl group with one or more unsaturations. In some embodiments, the hydrophobic block is one or more unsubstituted saturated C₅-C₂₀ alkyl group, optionally one or more unsubstituted saturated C₅-C₁₀ alkyl group. In an exemplary embodiment, the hydrophobic block is a group corresponding to C₆, C₉, C₁₀, C₁₂, C₁₄, C₁₆, C₁₈, or C₂₀ group. In some embodiments, the hydrophobic block is an unsubstituted alkyl group having at least 10 carbons, optionally for some applications at least 14 carbons and optionally for some applications at least 16 carbons.

In a specific embodiment, each of the semi-fluorinated block copolymers independently has the formula (FX1):

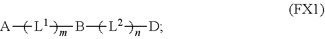

where A is the hydrophilic block, B is the fluorophilic block and D is the hydrophobic block; where L¹ and L² are each independently a linking group; and where m is 0 or 1 and n is 0 or 1. For embodiments where m is 0, L¹ is not present and A and B are directly bonded to one another. For embodiments where n is 0, L² is not present and B and D are directly bonded to one another. In some embodiments, the semi-fluorinated block copolymers independently have the formula (FX1), wherein m is 0. In some embodiments, the semi-fluorinated block copolymer independently has the formula (FX1), wherein n is 0.

In certain embodiments, A is —(CH₂CH₂O)_{q}R¹, where R¹ is hydrogen, methyl, C₁-C₁₀ alkyl, C₃-C₁₀ cycloalkyl, C₅-C₁₀ aryl, C₅-C₁₀ heteroaryl, C₁-C₁₀ alkoxy or C₁-C₁₀ acyl and q is an integer selected from the range of 10 to 300, and optionally for some applications 20 to 100 and optionally for some applications 20 to 50. In certain embodiments, B is —(CF₂)ₚ—, where p is an integer selected from the range of 3 to 20, and optionally for some embodiments 3 to 15 and optionally for some embodiments 6 to 15. In certain embodiments, D is —(CH₂)ₒR², where o is an integer selected from the range of 5 to 30, and optionally for some applications 5 to 20, and optionally for some applications 10 to 18, and in some applications is 16, and R² is hydrogen or C₁-C₅ alkyl.

In a specific embodiment, each of the semi-fluorinated block copolymers independently has the formula (FX2):

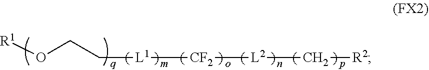

wherein q is a integer selected from the range of 10 to 300, o is an integer selected from the range of 6 to 15, and p is an integer selected from the range of 10 to 27; wherein R¹ is hydrogen, methyl, C¹-C¹⁰ alkyl, C³-C¹⁰ cycloalkyl, C⁵-C¹⁰ aryl, C⁵-C¹⁰ heteroaryl, C¹-C¹⁰ alkoxy or C¹-C¹⁰ acyl; wherein R² is hydrogen, halo or C¹-C⁵ alkyl; wherein each of L¹ and L² is independently null, —O—, —O(CH₂)ₑ—, —(CH₂)ₑ—, —(CH₂)ₑO(CH₂)_{f}—, —(CH₂)ₑS(CH₂)_{f}—, —(CH₂)ₑNR¹¹(CH₂)_{f}—, —(CH₂)ₑCONR¹²(CH²)_{f}—, —(CH₂)ₑCONR¹³(CH₂)_{f}—, —(CH₂)ₑNR¹⁴COO(CH₂)^{f}—, —(CH₂)ₑNR¹⁵CO(CH₂)_{f}— or —(CH₂)ₑNR¹⁶CONR¹⁷(CH₂)^{f}—; wherein each of R¹¹-R¹⁷ is independently hydrogen, methyl, or C₁-C₅ alkyl; and wherein each of e and f is independently an integer selected from the range of 0 to 5. In a specific embodiment, when e is 0, the (CH₂)ₑ group is not present and moieties adjacent in the above described structures for L¹ and L² are directly bonded to one another. In a specific embodiment, when f is 0, the (CH₂)_{f} group is not present and moieties adjacent to the (CH₂)_{f} group in the above described structures for L¹ and L² are directly bonded to one another.

In a specific embodiment, L¹ is —O—. In a specific embodiment, L² is —OCH₂—. In a specific embodiment, each of the semi-fluorinated block copolymers independently has the formula (FX3A) or (FX3B):

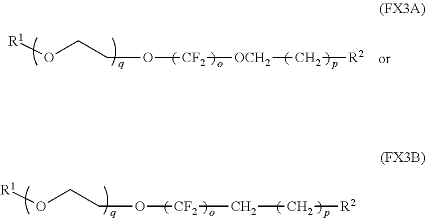

In an exemplary embodiment, each of the semi-fluorinated block copolymers independently has the formula (FX4A) or (FX4B):

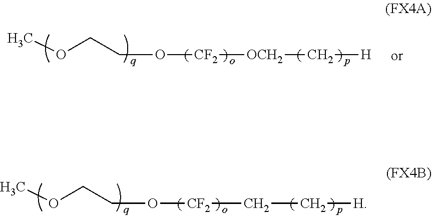

In one embodiment, the therapeutic emulsions of the invention may further comprise an imaging agent to form a theranostic emulsion. An imaging agent of the invention may include an MRI contrast agent. MRI is widespread for imaging due to its noninvasive nature, deep tissue penetration, excellent sensitivity, and widespread application. Though less common, ¹⁹F nuclei can be used easily for MRI with similar sensitivity to proton, 0.83. In the instant invention, fluorocarbons with high signal intensity can be used to image anatomical systems.

Accordingly, the instant invention may further comprise a fluorophilic imaging agent which is capable of physically associating with the semifluorinated block copolymers of the invention. The fluorophilic imaging agent, in one embodiment, physically associates with the fluorous or fluorophilic block as an intermediate “shell” in between the hydrophobic core and the hydrophilic exterior.

Thus, the positioning and delivery of the theranostic emulsions of the invention may be directly imaged using MRI visualization by utilizing the intrinsic properties of ¹⁹F nuclei via the incorporation of a fluorophilic imaging agent. The imaging agent is optionally an imaging agent which has a high signal intensity and low toxicity. High signal intensity occurs when a perfluorous compound exhibits a high number of identically electronically and/or magnetically situated fluorine atoms. Therefore, in one embodiment, the imaging agent includes perfluoroalkanes, perfluoroalkylamines, perfluoro-crown-ethers, perfluorinated alcohols, perfluorohaloalkanes, perfluorinated carboxylic acids, perfluorinated acrylates, and perfluorinated esters. In more preferred embodiments, the imaging agent comprises perfluoropolyether, perfluoro-15-crown-5-ether, perfluoro 12-crown-4 ether, perfluoro 18-crown-6 ether, sulfur hexafluoride, hexafluoroethane, or perfluoropropane. In one embodiment, perfluoro-15-crown-5-ether (PFCE) was selected as the fluorous contrast agent. This clear, organic oil is very stable under physiological conditions with a high boiling point. The structure of the macrocycle is thought to tend toward a puckered ring with high flexibility in solution leading to all twenty fluorine atoms exhibiting a single peak in ¹⁹F-NMR. Because the fluorine atoms are magnetically equivalent, this system is excellent for highly sensitive ¹⁹F-NMR and therefore ¹⁹F-MRI applications. In one embodiment of the present invention, the imaging agent is present in the emulsion at a concentration of between 5 mg ai^{l} to 750 mg mL⁻¹ in said emulsion, or between about 2 and 50% v/v, about 6 and 33% v/v.

In some embodiments, the therapeutic and theranostic emulsions of the present invention have improved properties relative to emulsions that lack the semi-fluorinated block copolymers of the invention and/or the imaging agent. Improved properties can include enhanced stability. The stability of an emulsion can be measured by following the particle size using dynamic light scattering. Particle ripening is very common in nanoemulsions and it is due to a variety of phenomena, one of the most important being Ostwald ripening, related to the difference in chemical potential between particles of different size. As seen in FIG. 4 herein, nanoemulsions of the present invention exhibit very minimal Ostwald ripening, in stark contrast to many of the nanoemulsions known in the art and on the market. Some minimal particle growth can be observed over time but as a whole the colloids display steady, consistent size. The nanoemulsion sizes observed for emulsions of the present invention are appropriate to take advantage of the EPR effect, e.g., the enhanced permeability and retention (EPR) effect where molecules of certain sizes (typically liposomes, nanoparticles, and macromolecular drugs) tend to accumulate in tumor tissue much more than they do in normal tissues up to 400 nm, and significantly below the FDA standard size cutoff for nanoparticles to be delivered in vivo, 500 nm. Not only that, but the emulsions of the present invention exhibit long-term stability required for consideration for in vivo treatment, up to one year or more. The addition of an imaging agent such as PFCE, and/or in combination with another fluorous excipient, results in increased colloidal stability and better drug retention. This added stability from the fluorinated excipient, without being bound by theory, may result from a thicker fluorous core surrounding the oil droplet, providing more control of the drug release, which is critical for the successful administration of chemotherapeutics. FIG. 4 shows that all compositions and emulsions incorporating the semi-fluorinated block copolymers of the invention have the same rate of ripening independently despite differing composition of emulsion particles. Thus, these emulsions have long term storage stability.

Drug release is a different property, which depends on the drug molecules' ability to migrate from emulsion into the body. As such, the release will be affected by an intermediate fluorous shell in the emulsion, due to the semi-fluorinated block copolymer. This intermediate fluorous shell will cause slower release of the hydrophobic drug, due to the fluorophobic nature of hydrophobic drugs. In certain embodiments, a perhalogenated fluorous compound, optionally MRI-active, provides delayed release of the therapeutic agent in the emulsions of the invention, so as to make stable emulsion-based formulations from a wide range of hydrophobic therapeutic agents. In embodiments, the structure, composition, size or concentration of the perhalogenated fluorous compound are selected so as to provide certain properties to the emulsion, such as physical properties, emulsion ripening rate, emulsion stability, therapeutic agent solubility, composition, toxicity, biocompatibility, therapeutic effectiveness, therapeutic agent delivery rate or release rate, immune or other physiological response or any combination of these. For example, in one embodiment, the perhalogenated fluorous compound is an MRI-active fluorous compound which also acts as a drug release delay agent. In one embodiment, for example, the perhalogenated fluorous compound is 5% to 30% by volume of the emulsion, optionally for some applications 5% to 20% by volume, and optionally for some applications 5% to 10% by volume.

The fluorous or fluorophilic imaging agent, and/or fluorous compound, which are optionally added to the emulsions, will also increase the effect of the fluorous shell and can further delay release of the hydrophobic drug. Such fluorous compounds can include a perhalogenated fluorous compound, as defined herein. Drug release pharmacokinetic profiles can be determined either in vivo or in vitro as known in the art, and the various amounts of each component of the emulsions of the invention may be adjusted to achieve a desired drug release profile.

Thus, in some embodiments, the therapeutic and in particular, the theranostic emulsions of the invention provides modulation and/or delay of drug release from the oil droplet core due to the presence of the surrounding fluorous shell. Delayed release may be measured, for example, by measuring a pharmacokinetic parameter such as the half-life of the therapeutic in the emulsions of the invention, either in vitro or in vivo via methods known in the art. Half-life may be improved at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 250%, at least 300%, at least 400%, at least 500%, at least 750%, or at least 1000% with respect to an emulsion which lacks the semi-fluorinated block copolymer and/or the imaging agent and/or a perhalogenated fluorous compound.

Selection of the perhalogenated fluorous compound, such as a perhalogenated fluorocarbon compound, having specific and well defined physical and chemical properties is also important in the present invention for providing therapeutic formulations providing enhanced delivery performance and stability, and for accessing therapeutic emulsions having clinically effective concentrations of hydrophobic compounds. In some embodiments, for example, the perhalogenated fluorous compound is provided that comprises a component of the dispersed droplet phase that controls the release rate of the hydrophobic compound from the droplets, thereby lowering the rate of droplet ripening processes such as Ostwald ripening. The perhalogenated fluorous compound of this aspect is useful for providing therapeutic emulsions, including nanoemulsions, exhibiting stable droplets sizes and/or comprising droplets that undergo growth at rates sufficiently low to allow their use as therapeutic agents.

First, perhalogenated fluorous compounds, such as perhalogenated fluorocarbon compounds, preferably exhibit high fluorophilicity. Exemplary perhalogenated fluorous compounds, such as perhalogenated fluorocarbon compounds, have a high affinity for the fluorous block of the semifluorinated block copolymer, which leads to a low interfacial tension with the block copolymer. For some applications, the number of fluorine-carbon bonds is an important parameter in selecting a perhalogenated fluorous compound, such as a perhalogenated fluorocarbon compound, having an appropriately high fluorophilicity. Perhalogenated fluorous compounds having between 12 to 25 carbon-fluorine bonds are desirable for some therapeutic formulations of the present invention. Alternatively, the number of carbon-fluorine bonds of the perhalogenated fluorous compound may be appropriately matched or otherwise related to the number of carbon-fluorine bonds of the fluorophilic block of the semi-fluorinated block copolymers.

Second, perhalogenated fluorous compounds, such as perhalogenated fluorocarbon compounds, preferably exhibit low solubility in water. Selection of perhalogenated fluorous compounds with low water solubility is useful for avoiding degradation of the present therapeutic emulsion caused by over-ripening of fluorinated therapeutic containing particles dispersed in a continuous aqueous phase. In an embodiment, the perhalogenated fluorous compound, such as a perhalogenated fluorocarbon compounds, has a solubility in water less than or equal to 20 nanomolar. The particle ripening rate depends on the solubility of the additive. Accordingly, use of perfluorooctyl bromide (abbreviated as PFOB), which has a solubility of 5 nM, provides for slow ripening. In principle, however, an perhalogenated fluorous compound that is more water-soluble, for example 20 nM, will also slow the ripening but not as much as fluoroderivatives that are less soluble.

Third, perhalogenated fluorous compounds, such as perhalogenated fluorocarbon compounds, useful in the present therapeutic formulations are preferably chemically inert. Perfluorinated compounds, bromine substituted perfluorinated compounds and chlorine substituted perfluorinated compounds provide useful chemically inert perhalogenated fluorocarbon compounds in the present invention.

Fourth, perhalogenated fluorous compounds, such as perhalogenated fluorocarbon compounds, useful in the present therapeutic formulations preferably are rapidly excreted, for example having a circulatory half-time (i.e., the time for the concentration of perhalogenated fluorous compound to decrease by half in the circulation) less than two weeks.

Fifth, perhalogenated fluorous compounds, such as perhalogenated fluorocarbon compounds, useful in the present therapeutic formulations preferably have a molecular weight selected over the range of 460 amu to 920 amu. Perhalogenated fluorous compounds having molecular weights below this range are typically susceptible to having too high a vapor pressure, which can lead to lung emphysema and other pulmonary complications. Perhalogenated fluorous compounds having molecular weights above this range typically exhibit excretion times that are undesirably long.

Sixth, perhalogenated fluorous compounds, such as perhalogenated fluorocarbon compounds, useful in the present therapeutic formulations preferably are provided as high purity reagents. Benefits of the use of high purity reagents are that no toxicity, carcinogenic, mutagenic, teratogenic effects, or immunological reactions, have been reported for many fluorocarbons when provided in a sufficiently pure form and chosen within an appropriate molecular weight range.

A variety of perhalogenated fluorous compounds are useful with the emulsions of the invention. In one embodiment, the perhalogenated fluorous compound has 12 to 25 carbon-fluorine bonds, optionally for some applications 15 to 20 carbon-fluorine bonds. In an exemplary embodiment, the perhalogenated fluorous compound is a substituted or unsubstituted fluorocarbon having a length of 4 to 20 carbons, and optionally for some applications 5 to 15 carbons, and optionally for some applications 5 to 10 carbons. In embodiments, the perhalogenated fluorous compound comprises a perfluorocarbon. In embodiments, the perhalogenated fluorous compound comprises a perfluorocarbon in which one or more fluorine atoms are substituted with Cl, Br or I, optionally in which one or more fluorine atoms are substituted with Cl or Br. In various embodiments, the substituted or unsubstituted fluorocarbon is linear, branched or cyclic. In embodiments, the substituted or unsubstituted fluorocarbon is a substituted or unsubstituted C₄-C₂₀ fluoroalkane, optionally for some applications C₄-C₁₅ fluoroalkane, and optionally for some applications C₄-C₁₀ fluoroalkane.

In embodiments, for example, the perhalogenated fluorous compound has a solubility in water less than or equal to 20 nanomolar. In embodiments, for example, the perhalogenated fluorous compound has a molecular weight selected over the range of 460 amu to 920 amu. In exemplary embodiments, the perhalogenated fluorous compound is selected from the group consisting of: a perfluorocarbon; a bromine substituted perfluorocarbon; a chlorine substituted perfluorocarbon; and a bromine and chlorine substituted perfluorocarbon.

A variety of specific perhalogenated fluorous compounds are useful with the emulsions of the invention. In specific embodiments, for example, the perhalogenated fluorous compound is selected from the group consisting of perfluorooctyl bromide, perfluorononyl bromide, perfluorodecyl bromide, perfluorodecalin, perfluorodichlorooctane, bis-perfluorobutyl ethylene and perfluoro(methyldecalin). In embodiments, the perhalogenated fluorous compound is perfluorooctyl bromide or perfluorodecalin. In embodiments, the perhalogenated fluorous compound is perfluorooctyl bromide.

Optionally, emulsions of the invention further comprise one or more additional perhalogenated fluorous compounds including, for example, any of the perhalogenated fluorous compounds disclosed herein. Optionally, emulsions of the invention further comprise one or more additional fluorocarbon compounds including, for example, any of the fluorocarbon compounds herein. Emulsions of the invention include formulations comprising two or more of any of the perhalogenated fluorous compounds disclosed herein, for example, the combination of perfluorooctyl bromide and perfluorodecalin.

A variety of therapeutic agents are useful with the emulsions of the invention. In certain embodiments, the structure, composition, size or concentration of the therapeutic agent is selected so as to make a stable emulsion-based formulation. In embodiments, the structure, composition, size or concentration of the therapeutic agent are selected so as to provide certain properties to the emulsion, such as physical properties, emulsion ripening rate, emulsion stability, therapeutic agent solubility, composition, toxicity, biocompatibility, therapeutic effectiveness, therapeutic agent delivery rate or release rate, immune or other physiological response or any combination of these. In an embodiment, for example, the therapeutic agent has a concentration of at least 0.1 mg mL⁻¹ in the emulsion, and optionally for some embodiments at least 1 mg mL⁻¹. In an embodiment, the therapeutic agent has a concentration selected from the range of 0.1 mg mL⁻¹ to 5 mg mL⁻¹ in the emulsion.

In certain embodiments, the therapeutic agent is a hydrophobic compound. Use of hydrophobic therapeutic agents is beneficial as a variety of hydrophobic therapeutic agents exhibit reduced toxicity, increased therapeutic effectiveness or smaller required therapeutic dosages as compared to some non-hydrophobic therapeutic agents. In addition, therapeutic agents for a desired clinical application may only be available as a hydrophobic compound. In embodiments, emulsions of the invention are, thus, particularly useful for providing a therapeutically deliverable quantity of a hydrophobic therapeutic agent in order to achieve a desired clinical outcome, such as treatment of cancer or fungal infection. In a specific embodiment, the hydrophobic compound has a concentration of 0.1 to 5 mg mL⁻¹, optionally for some applications a concentration of 0.5 to 2 mg mL⁻¹.

In a specific embodiment, the hydrophobic compound is noncovalently associated with the hydrophobic block of the semi-fluorinated block copolymers. For example, in embodiments, the hydrophobic compound is dissolved or solvated in a hydrophobic liquid which is also associated with the hydrophobic block, such that the hydrophobic compound is suspended or otherwise deliverable by an emulsion of the invention.

A variety of hydrophobic compounds are useful with the emulsions of the invention. In a specific embodiment, the hydrophobic compound is a hydrophobic drug. In one embodiment, the hydrophobic drug is an antifungal drug, for example, a polyene antifungal such as, for example, candicidin, filipin, hamycin, natamycin, nystatin, rimocidin; an imidazole antifungal, such as, for example, bifonazole, butoconazole, clotrimazole, econazole, fenticonazole, isoconazole, ketoconazole, luliconazole, miconazole, omoconazole, oxiconazole, sertaconazole, sulconazole, tioconazole; a triazole antifungal such as albaconazole, efinaconazole, epoxiconazole, fluconazole, isavuconazole, itraconazole, posaconazole, propiconazole, ravuconazole, terconazole, voriconazole; and others, such as thiazoles, griseofulvin, among others.

In another embodiment, the hydrophobic drug is a hydrophobic anticancer drug, such as, for example, paclitaxel, doxorubicin, retinoic acid series, camptothecin, docetaxel, tamoxifen, anasterozole, topotecan, belotecan, irinotecan, gleevec and vincristine, among others.

As described above, the present invention provides emulsions, such as emulsions comprising a continuous phase and a dispersed phase. In certain embodiments, the aqueous solution of the continuous phase comprises a saline solution. In embodiments, for example, the aqueous solution of the continuous phase is isotonic to blood plasma. In an embodiment, the dispersed phase comprises a plurality of droplets dispersed in the continuous phase. In embodiments, for example, the droplets dispersed in the continuous phase comprise self-assembled supramolecular structures. Various emulsion embodiments do not include micelle-based solutions, but instead comprise droplets of the dispersed phase suspended in the continuous phase.

In a specific embodiment, an exemplary emulsion of the invention comprises the hydrophobic compound at a concentration of 0.2 to 80 mg mL⁻¹ in the oil phase, the perhalogenated fluorous compound or the perfluorinated compound (imaging agent) is 5% to 35% by volume of the emulsion; and the semi-fluorinated block copolymers at a concentration selected from the range of 5 to 50 mg mL⁻¹. In a specific embodiment, an exemplary emulsion of the invention comprises the hydrophobic compound at a concentration of 0.2 to 80 mg mL⁻¹ in the oil phase; and the semi-fluorinated block copolymers at a concentration selected from the range of 5 to 50 mg mL⁻¹. Exemplary emulsion embodiments are useful for administration to a patient in need thereof via intravenous injection.

In embodiments, the hydrophobic compound is at a concentration of 0.2 to 80 mg ai^{l}, or about 1 to 70 mg mL⁻¹, or about 5 to 60 mg mL⁻¹, or about 10-50 mg ai^{l}, or about 20 to 40 mg mL⁻¹, in the oil phase. In embodiments, the perhalogenated fluorous compound or the perfluorinated compound (imaging agent) is 5% to 35% by volume of the emulsion; is 10% to 40% by volume of the emulsion, is 15% to 35% by volume of the emulsion, is 20% to 30% by volume of the emulsion. In embodiments, the semi-fluorinated block copolymers, in the emulsion, are at a concentration selected from the range of 10 to 45 mg mL⁻¹, at a concentration selected from the range of 15 to 40 mg mL⁻¹ at a concentration selected from the range of 20 to 35 mg mL⁻¹, or at a concentration selected from the range of 25 to 30 mg mL⁻¹.

Emulsions of embodiments of the invention are stable and possess a shelf life such that the emulsions do not quickly settle into two or more phases and thus are suitable for administration to a patient or subject at a time period after the emulsion is prepared or manufactured, such as a time period greater than 1 day or greater than 1 month, optionally greater than 6 months or optionally up to one year. In an exemplary embodiment, the droplets do not undergo an appreciable change in size over a period of 1 day to 52 weeks, from 1 week to 40 weeks, from 4 weeks to 30 weeks, from 8 weeks to 24 weeks, for example. Alternatively, an appreciable change in size does not occur for at least 1 week, at least 4 weeks, at least 8 weeks, at least 12 weeks, at least 16 weeks, at least 24 weeks, at least 28 weeks, at least 32 weeks, at least 36 weeks, at least 40 weeks, at least 44 weeks, at least 48 weeks, at least 52 weeks, at least 60 weeks, at least 68 weeks, at least 76 weeks, at least 84 weeks, at least 92 weeks, or longer. In embodiments, for example, an appreciable change in size is a change that is greater than or equal to a 10% increase.

For certain embodiments, an emulsion of the invention comprises a nanoemulsion. For example, in some embodiments, the emulsion comprises droplets having an average diameter selected from the range of 1 nm to 500 nm. For example, in some embodiments, the emulsion comprises droplets having an average diameter less than 1000 nm. In some embodiments, the emulsion comprises droplets having an average diameter less than 400 nm. For some embodiments, the emulsion does not comprise micelles. For some embodiments, the emulsion does not comprise vesicles. In certain embodiments, the emulsion is not a microemulsion. For some embodiments, the emulsion comprises a supramolecular structure. For other embodiments, the emulsion does not comprise a supramolecular structure. Adjustments in size of the droplets in the nanoemulsions can be adjusted by methods known in the art, such as pressure adjustments in a microfluidizer. In some embodiments, an individual droplet may have a size of from about 70 nm to about 300 nm. For imaging applications, the size may be from about 150 to about 300 nm. For theranostic applications, the size may be smaller, from about 100 to 120 nm.

In another aspect, the present invention provides a method of delivering a therapeutic agent and/or an imaging agent to a patient in need thereof. In an embodiment, such a method comprises, for example, the steps of providing an oil in water emulsion comprising a hydrophobic liquid, an aqueous solution, and semi-fluorinated block copolymers according to the invention; and optionally a therapeutic agent or imaging agent according to the invention. In embodiments, where a therapeutic agent is present, the therapeutic agent is released from the emulsion, thereby delivering the therapeutic agent to the patient in need thereof.

In exemplary embodiments, an emulsion administered to a patient comprises any of the emulsions described previously herein. In a specific embodiment, an emulsion administered to a patient is a nanoemulsion. In a specific embodiment, the hydrophobic compound administered to a patient is a hydrophobic drug.

In exemplary embodiments of methods of this aspect, the step of administering the emulsion provides for controlled release of the hydrophobic drug from the emulsion. Optionally, the step of administering the emulsion is carried out via intravenous injection. In a specific embodiment, a volume of the emulsion less than or equal to 500 mL is administered to the patient. In exemplary embodiments, a volume of the emulsion selected from the range 0.1 mL to 500 mL is administered to the patient. In a specific embodiment, the emulsion is delivered to the patient at a rate less than or equal to 100 mL per minute. In exemplary embodiments, the emulsion is delivered to the patient at a rate selected from the range of 0.01 to 10 mL per minute.

In a further aspect, provided are methods of making emulsions. In exemplary embodiments, the emulsion is a nanoemulsion. An exemplary method of this aspect comprises the steps of: providing a therapeutic formulation or a theranostic formulation according to the invention; and emulsifying the therapeutic or theranostic formulation, thereby making an emulsion according to the invention.

In certain embodiments of methods of this aspect, the step of emulsifying the therapeutic formulation comprises the steps of: adding the hydrophobic compound to the oil and allowing to fully solubilize by methods known in the art. The solubilized hydrophobic compound may be added to the semi-fluorinated block copolymer and optional imaging agent and energy may be added, such as by homogenization at high speed. Optionally, a further mixing step such as a microfluidizer may be added. Optionally, the resultant emulsion can be filtered with a filter, such as a 0.45 micron nylon filter. Thus, in exemplary embodiments, methods of this aspect further comprise a step of lowering a temperature of the mixture during the step of homogenizing the mixture. In embodiments, for example, the step of homogenizing the mixture is carried out using a lower energy mixer, a microfluidizer or both. Low-energy mixers are known in the art, such as described in AlChE J., 57: 27-39. doi: 10.1002/aic.12253.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 provides a first scheme for synthesis of M2F8H18 amphiphile.

FIG. 2 provides a second synthesis scheme for an alternative, shortened synthesis of M2F8H18 amphiphile.

FIG. 3 provides a proposed therapeutic nanoemulsion structure, along with the chemical formula of a particular embodiment of the semifluorinated block copolymer.

FIG. 4 shows change in particle size of M2F8H18 nanoemulsions over time as measured by DLS: (diamond) 4 mL medium chain triglycerides (MCT)only, (triangle) 2 mL MCT and 1 mg/mL paclitaxel, (circle) 4 mL MCT and 4 mg/mL paclitaxel, (darker triangle) 2 mL MCT and 10% v/v PFCE, (square) MCT, 1 mg/mL paclitaxel, and 23.5% v/v PFCE. All nanoemulsions prepared in 0.9% w/w NaCl.

FIG. 5 shows nanoemulsion drug release profiles: (squares) M2F8H18 and MCT only, (circles) M2F8H18 with MCT and PFCE.

FIG. 6 shows A549 cell viability studies: (white column) non-treated control, (second column) 0.05% DMSO control, (3^{rd}-5^{th} column) M2F8H18 nanoemulsion with no drug and MCT, (6^{th} through 11^{th} column) M2F8H18 nanoemulsion loaded with 4 mg/mL paclitaxel and MCT, (12^{th} through 17^{th} column) paclitaxel standard solutions.

FIG. 7 shows zebrafish survival rates after exposure for 96 (hours post fertilization). (hpf): non-treated control (far left column); and for each concentration (30 μM, 111 μM, 333 μM, and 1 mM) of each of perfluoro-octanoic acid, (left) M2F8H18 polymer (right).

FIG. 8 shows ¹H-MR (left) and ¹⁹F-MR (right) phantom images taken at 4.7 T. Image background threshold was adjusted to only view above 5% voxels. From top-center moving clockwise, the concentration of PFCE in the M2F8H18 nanoemulsion is 154 mM, 61 mM, 31 mM, 3 mM, and 0 mM. Strong signal intensity can be viewed at 31 mM while 3 mM is below the limit of detection.

FIG. 9 shows cryogenic-electron microscopy (Cryo-EM) images of the nanoemulsions. The dark oil in the core is the perfluoro-15-crown-5-ether; the light oil in the core is the medium chain triglycerides.

### STATEMENTS REGARDING CHEMICAL COMPOUNDS AND NOMENCLATURE

In an embodiment, a composition or compound of the invention is isolated or purified. In an embodiment, an isolated or purified compound is at least partially isolated or purified as would be understood in the art. In an embodiment, the composition or compound of the invention has a chemical purity of 95%, optionally for some applications 99%, optionally for some applications 99.9%, optionally for some applications 99.99%, and optionally for some applications 99.999% pure.

Many of the molecules disclosed herein contain one or more ionizable groups. Ionizable groups include groups from which a proton can be removed (e.g., —COOH) or added (e.g., amines) and groups which can be quaternized (e.g., amines). All possible ionic forms of such molecules and salts thereof are intended to be included individually in the disclosure herein. With regard to salts of the compounds herein, one of ordinary skill in the art can select from among a wide variety of available counterions that are appropriate for preparation of salts of this invention for a given application. In specific applications, the selection of a given anion or cation for preparation of a salt can result in increased or decreased solubility of that salt.

As used throughout the present description, the expression “a group corresponding to” an indicated species expressly includes a radical (including a monovalent, divalent and trivalent radical) derived from that species.

The compounds of this invention and used with the methods or emulsions of the invention can contain one or more chiral centers. Accordingly, this invention is intended to include racemic mixtures, diasteromers, enantiomers, tautomers and mixtures enriched in one or more stereoisomer. The scope of the invention as described and claimed encompasses the racemic forms of the compounds as well as the individual enantiomers and non-racemic mixtures thereof.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

As used herein, the term “substituted” refers to a compound wherein a hydrogen is replaced by another functional group.

As is customary and well known in the art, hydrogen atoms in formulas (FX1)-(FX4) are not always explicitly shown, for example, hydrogen atoms bonded to the carbon atoms of aromatic, heteroaromatic, and alicyclic rings are not always explicitly shown in formulas (FX1)-(FX4). The structures provided herein, for example in the context of the description of formulas (FX1)-(FX4), are intended to convey to one of reasonable skill in the art the chemical composition of compounds of the methods and compositions of the invention, and as will be understood by one of skill in the art, the structures provided do not indicate the specific positions of atoms and bond angles between atoms of these compounds.

As used herein, the terms “alkylene” and “alkylene group” are used synonymously and refer to a divalent group derived from an alkyl group as defined herein. The invention includes compounds having one or more alkylene groups. Alkylene groups in some compounds function as attaching and/or spacer groups. Compounds of the invention may have substituted and/or unsubstituted C₁-C₂₀ alkylene, C₁-C₁₀ alkylene and C₁-C₅ alkylene groups.

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as attaching and/or spacer groups. Compounds of the invention may have substituted and/or unsubstituted C₃-C₂₀ cycloalkylene, C₃-C₁₀ cycloalkylene and C₃-C₅ cycloalkylene groups.

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as attaching and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups.

As used herein, the terms “heteroarylene” and “heteroarylene group” are used synonymously and refer to a divalent group derived from a heteroaryl group as defined herein. The invention includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as attaching and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups.

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as attaching and/or spacer groups. Compounds of the invention include substituted and/or unsubstituted C₂-C₂₀ alkenylene, C₂-C₁₀ alkenylene and C₂-C₅ alkenylene groups.

As used herein, the terms “cylcoalkenylene” and “cylcoalkenylene group” are used synonymously and refer to a divalent group derived from a cylcoalkenyl group as defined herein. The invention includes compounds having one or more cylcoalkenylene groups. Cycloalkenylene groups in some compounds function as attaching and/or spacer groups. Compounds of the invention include substituted and/or unsubstituted C₃-C₂₀ cylcoalkenylene, C₃-C₁₀ cylcoalkenylene and C₃-C₅ cylcoalkenylene groups.

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as attaching and/or spacer groups. Compounds of the invention include substituted and/or unsubstituted C₂-C₂₀ alkynylene, C₂-C₁₀ alkynylene and C₂-C₅ alkynylene groups.

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy)ₙ-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenylgroups can also carry alkyl groups. Cycloalkenylgroups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms.

Aryl groups include groups having one or more 5-, 6- or 7- member aromatic rings, including heterocyclic aromatic rings. The term heteroaryl specifically refers to aryl groups having at least one 5-, 6- or 7- member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms.

As to any of the groups described herein which contain one or more substituents, it is understood that such groups do not contain any substitution or substitution patterns which are sterically impractical and/or synthetically non-feasible. In addition, the compounds of this invention include all stereochemical isomers arising from the substitution of these compounds. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including —CN;

—COOR where R is a hydrogen or an alkyl group or an aryl group and more specifically where R is a methyl, ethyl, propyl, butyl, or phenyl group all of which groups are optionally substituted;
—COR where R is a hydrogen or an alkyl group or an aryl group and more specifically where R is a methyl, ethyl, propyl, butyl, or phenyl group all of which groups are optionally substituted;
—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;
—SR, where R is hydrogen or an alkyl group or an aryl group and more specifically where R is hydrogen, methyl, ethyl, propyl, butyl, or a phenyl group, which are optionally substituted;
—SO₂R, or —SOR where R is an alkyl group or an aryl group and more specifically where R is a methyl, ethyl, propyl, butyl, or phenyl group, all of which are optionally substituted;
—OCOOR where R is an alkyl group or an aryl group;
—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;
—OR where R is H, an alkyl group, an aryl group, or an acyl group all of which are optionally substituted. In a particular example R can be an acyl yielding
—OCOR″ where R″ is a hydrogen or an alkyl group or an aryl group and more specifically where R″ is methyl, ethyl, propyl, butyl, or phenyl groups all of which groups are optionally substituted; and

### —NO₂.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible. In addition, the compounds of this invention include all stereochemical isomers arising from the substitution of these compounds.

Pharmaceutically acceptable salts comprise pharmaceutically-acceptable anions and/or cations. As used herein, the term “pharmaceutically acceptable salt” can refer to acid addition salts or base addition salts of the compounds in the present disclosure. A pharmaceutically acceptable salt is any salt which retains at least a portion of the activity of the parent compound and does not impart significant deleterious or undesirable effect on a subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, −32-cilexetil, citric, edetic, edisylic, estolic, esyl, esylic, formic, fumaric, gluceptic, gluconic, glutamic, glycolic, glycolylarsanilic, hexamic, hexylresorcjnoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulf uric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Pharmaceutically acceptable salts may be derived from amino acids, including but not limited to cysteine. Other pharmaceutically acceptable salts may be found, for example, in Stahl et al., Handbook of Pharmaceutical Salts: Properties, Selection, and Use, Wiley-VCH; Verlag Helvetica Chimica Acta, Zurich, 2002. (ISBN 3-906390-26-8). Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl⁻, Br⁻), sulfate, acetates (e.g., acetate, trifluoroacetate), ascorbates, aspartates, benzoates, citrates, and lactate.

The compounds of this invention can contain one or more chiral centers. Accordingly, this invention is intended to include racemic mixtures, diasteromers, enantiomers, tautomers and mixtures enriched in one or more stereoisomer. The scope of the invention as described and claimed encompasses the racemic forms of the compounds as well as the individual enantiomers and non-racemic mixtures thereof.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Supramolecular structure” refers to structures comprising an assembly of molecules. Supramolecular structures include assemblies of molecules, such as linear block copolymers having hydrophilic, fluorophilic, and hydrophobic blocks, which are selectively oriented such that hydrophilic portions of the molecules are oriented outward toward a continuous aqueous phase, hydrophobic portions form an inner core and fluorophilic portions of the molecules are oriented in between to form a fluorous shell. Supramolecular structures include, but are not limited to, droplets, emulsions, encapsulated droplets. Supramolecular structures of the present invention include self-assembled structures. Supramolecular structures may comprise the dispersed phase of a colloid, such as an emulsion or nanoemulsion.

“Semi-fluorinated” refers to chemical compounds having at least one fluorine atom, for example molecules having at least one carbon-fluorine bond.

Fluorocarbons as used herein refer to chemical compounds that contain at least one carbon-fluorine bond.

“Perfluorinated” and “perfluorocarbon” refers to chemical compounds that are analogs of hydrocarbons wherein all hydrogen atoms in the hydrocarbon are replaced with fluorine atoms. Perfluorinated molecules can also contain a number of other atoms, including bromine, chlorine, and oxygen. A bromine substituted perfluorocarbon is a perfluorocarbon wherein one or more of the fluorine atoms have been replaced with a bromine atom. A chlorine substituted perfluorocarbon is a perfluorocarbon wherein one or more of the fluorine atoms have been replaced with a chlorine atom. A chlorine and bromine substituted perfluorocarbon is a perfluorocarbon wherein one or more of the fluorine atoms have been replaced with a chlorine atom and wherein one or more of the fluorine atoms have been replaced with a bromine atom.

“Perhalogenated fluorous compound” refers to fluorophilic chemical compounds that are analogs of a substituted or unsubstituted hydrocarbon wherein the hydrogen atoms are replaced with halogen atoms, such as fluorine, chlorine and bromine. Perhalogenated fluorous compounds can also contain a number of other atoms, including oxygen, sulfur and nitrogen. Perhalogenated fluorous compounds include perfluorocarbons and substituted perfluorocarbons, such as chlorine substituted perfluorocarbons, bromine substituted perfluorocarbons and chlorine and bromine substituted perfluorocarbons.

“Emulsion” refers to a mixture of two or more immiscible substances, such as a mixture of two immiscible liquids. Emulsions are a type of colloid that comprise at least one dispersed phase dispersed in a continuous phase. Emulsions are broadly defined as two immiscible phases in which a first phase is dispersed within a second phase, such as a two-phase system in which one liquid is dispersed throughout a second liquid in the form of small droplets. The two phases of an emulsion are generally referred to as the continuous phase and the dispersed phase, with the dispersed phase typically present as a smaller volume percentage. A dispersion of oil in water is referred to as an oil-in-water (o/w) emulsion. For o/w emulsions the emulsifying agent is typically more soluble in the aqueous phase. The reverse emulsion, water-in-oil, is abbreviated w/o and is stabilized by surfactants that are more stable in the oil phase. In an aqueous emulsion, the continuous phase is an aqueous solution.

Emulsions are not thermodynamically stable, but the stability can be improved by additives such as surfactants. As non-equilibrium systems, the formation of nanoemulsions generally requires an input of energy. High-energy emulsification methods commonly involve the introduction of mechanical shear through such equipment as high-shear stirrers, high-pressure homogenizers, microfluidizers or ultrasound generators. A microfluidizer is the piece of equipment used in the pharmaceutical industry for the production of emulsions that works by dividing a stream of liquid into two parts, passing each through a narrow opening and then colliding the streams under high pressure. The high shear forces created by the collision provide very fine emulsions with generally narrow particle size distributions. In typical usage, a coarse emulsion (diameter >1 μm) is first formed by some other method, and the size of that larger emulsion is reduced in the microfluidizer. The final droplet size and distribution shape will be dependent upon both the emulsion components (surfactant amount, oil volume percent, etc.) and the processing parameters (time, temperature, pressure etc.). As the desired droplet size decreases, the energy required for formation increases. Ultrasonic emulsification is also effective to reduce the size of emulsion droplets into the nanoscale. Emulsions can also be formed by changing the temperature of a mixture of immiscible liquids, for example by rapid cooling or heating to produce kinetically stable emulsions with small droplet sizes and narrow size distributions.

Emulsions include nanoemulsions comprising nanoscale droplets of one immiscible liquid dispersed within another. As used herein a nanoemulsion is a heterogeneous system composed of one immiscible liquid dispersed as droplets within another liquid, where the average droplet diameter is below 1000 nm.

“Flocculation” refers to a process in which clusters of two or more droplets behave kinetically as a unit, but individual droplets still maintain their identity. Flocculation may be reversible, or lead to coalescence, which is irreversible.

“Coalescence” is the collision, and subsequent irreversible fusion, of two droplets. The ultimate end of coalescence is complete phase separation. Flocculation precedes coalescence, so the same methods that are appropriate for prevention of flocculation also prevent coalescence. A thick, surfactant film adsorbed at the interface is often sufficient to prevent coalescence, whether in nano- or macro-emulsions.

“Ostwald ripening” refers to the growth in the size of emulsion droplets as the contents of one drop diffuse into another. The driving force for this growth is the difference in chemical potential between droplets, which is generally not substantial for droplets larger than 1 μm. Therefore, Ostwald ripening primarily affects nanoemulsions, and is an important factor for nanoemulsions for therapeutic applications.

“Polymer” refers to a molecule comprising a plurality of repeating chemical groups, typically referred to as monomers. A “copolymer”, also commonly referred to as a heteropolymer, is a polymer formed when two or more different types of monomers are linked in the same polymer. “Block copolymers” are a type of copolymer comprising blocks or spatially segregated domains, wherein different domains comprise different polymerized monomers. In a block copolymer, adjacent blocks are constitutionally different, i.e. adjacent blocks comprise constitutional units derived from different species of monomer or from the same species of monomer but with a different composition or sequence distribution of constitutional units. Different blocks (or domains) of a block copolymer may reside on different ends of a polymer (e.g. [A][B]), or may be provided in a selected sequence ([A][B][A][B]). “Diblock copolymer” refers to block copolymers having two different chemical blocks. “Triblock copolymer” refers to block copolymers having three different chemical blocks. Polymers of the present invention include block copolymers having a first block comprising a larger polymer (e.g., 10-300) such as a PEG polymer having 10 to 270 monomers smaller polymer (e.g., 2 to 30 monomers), an intermediate block such as a fluorocarbon, including but not limited to, a fluorocarbon such as a fluorinated or perfluorinated alkane, and a third interior hydrophobic block. Block copolymers of the present invention are capable of undergoing self-assembly to make supramolecular structures, such as encapsulated droplets. As used herein, the term block copolymer includes compositions comprising a first block comprising a PEG polymer conjugated to a second block comprising perfluorinated or semifluorinated molecular domain, such as a perfluorinated or semifluorinated alkane or a perfluorinated or semifluorinated tail and further conjugated to a third block comprising a hydrophobic polymer. As used herein, the term block copolymer also includes functionalized block copolymers, such as copolymers having additional moieties for targeting a supramolecular structure to an active site, for stabilizing a supramolecular structure or for selecting the release kinetics of a supramolecular structure containing a fluorinated therapeutic compound.

As used herein “hydrophilic” refers to molecules and/or components (e.g., functional groups, blocks of block polymers, etc.) of molecules having at least one hydrophilic group, and hydrophobic refers to molecules and/or components (e.g., functional groups of polymers, and blocks of block copolymers etc.) of molecules having at least one hydrophobic group. Hydrophilic molecules or components thereof tend to have ionic and/or polar groups, and hydrophobic molecules or components thereof tend to have nonionic and/or nonpolar groups. Hydrophilic molecules or components thereof tend to participate in stabilizing interactions with an aqueous solution, including hydrogen bonding and dipole-dipole interactions. Hydrophobic molecules or components tend not to participate in stabilizing interactions with an aqueous solution and, thus often cluster together in an aqueous solution to achieve a more stable thermodynamic state. In the context of block copolymers of the present invention, a hydrophilic block is more hydrophilic than a hydrophobic group of an amphiphilic block copolymer, and a hydrophobic group is more hydrophobic than a hydrophilic block of an amphiphilic polymer.

As used herein “fluorophilic” refers to molecules and/or components (e.g., functional groups, blocks of block polymers etc.) of molecules having at least one fluorophilic group. A fluorophilic group is one that is capable of participating in stabilizing interactions with a fluorous phase. Fluorophilic groups useful in block copolymers compounds of the invention include, but are not limited to, fluorocarbon groups, perfluorinated groups and semifluorinated groups.

As used herein “hydrophobic” refers to molecules and/or components (e.g., functional groups, blocks of block polymers etc.) of molecules having at least one hydrophobic group. A hydrophobic group can be understood as a group that is repelled from water. Hydrophobic groups tend to be nonpolar and do not form hydrogen bonds. While hydrophobic materials are usually lipophilic, silicones and fluorocarbons are not lipophilic. In embodiments, the hydrophobic materials of the invention are lipophilic, which generally refers to the material's ability to dissolve in, or dissolve, fats, oils, lipids and hydrophobic/lipophilic compounds. Hydrophobic blocks are known in the art, and include such materials as one or more linear or branched C₅-C₂₀ alkyl block, a poly (ϵ-caprolactone) block, a poly(lactic acid) block; a poly(propylene glycol) block; a poly(amino acid) block; a poly(ester) block and poly(lactic-co-glycolic acid) block, polydimethylsiloxane (PDMS) block, poly(caprolactone (PCL) block, poly(methyl methacrylate) (PMMA), and the like.

In the context of the present invention the term patient is intended to include a mammalian subject, such as a human, as well as a subject such as an animal, such as a companion or food animal.

Before the present methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

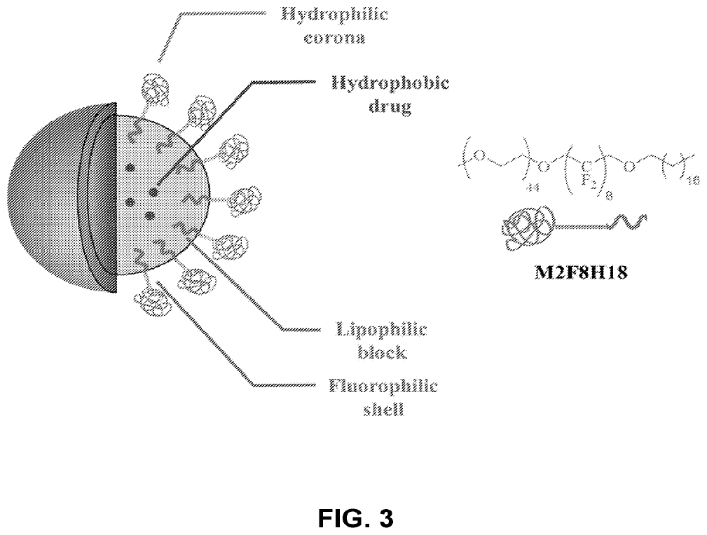

FIG. 3 provides a schematic illustration of the formation a supramolecular structure in an emulsion comprising a hydrophobic drug in a hydrophobic core, and semi-fluorinated block copolymers comprising a hydrophilic block, a fluorophilic block, and a hydrophobic block. Without being bound by any theory, FIG. 3 is provided to illustrate aspects of the invention relating to one potential structure of the dispersed phase droplets in emulsions and formulations of the invention, and is not intended to limit the structure of the dispersed phase droplets. As will be understood by one of skill in the art FIG. 3 is merely a schematic representation to convey an understanding of the components of the present emulsions, and is not intended to provide the actual structures, physical dimensions and relative arrangement of the components of the dispersed droplet phase.

The invention may be further understood by the following non-limiting examples.

### Example 1

### Emulsion-Based Formulations

This example provides a description of compositions and physical properties of specific examples of emulsions useful in the present formulations and therapeutic methods, which taken together demonstrate useful properties and applications of certain embodiments of the present invention.

An example of a hydrophobic drug model is the chemotherapeutic, paclitaxel. This antimitotic drug was selected for its high potency against many cancer types including breast, ovarian, and lung cancer as well as Kaposi's carcinoma. Paclitaxel functions by the promotion of tubulin polymerization and equimolar binding to tubulin dimers, resulting in mitotic arrest. Unfortunately, this molecule exhibits very poor water solubility and pharmacokinetics in vivo. Due to these challenging characteristics, paclitaxel was first administered intravenously as Taxol® in combination with Cremophor EL®, a nonionic surfactant with polyoxyethylated castor oil, and ethanol. Sadly, Taxol® leads to widespread hypersensitivity reactions, plasticizer leaching, and even neuropathy due to the solvent mixture. An additional paclitaxel formulation, Abraxane®, was approved in 2005 involving albumin-bound paclitaxel nanoparticles. Despite improvements in side effect toxicity, this formulation still lacks high potency due to the discrepancy between injected drug and drug that actually reaches the tumor. The need for an improved delivery method for this highly cytotoxic drug is still present.

Materials and Methods

1H,1H,10H,10H-perfluorodecane-1,10-diol was purchased from SynQuest Laboratories Inc. (Alachua, Fla.) and perfluoro-15-crown-5-ether was purchased from Oakwood Chemical (Estill, S.C.). Normal saline (AirLife sterile 0.9% sodium chloride for irrigation USP) was purchased from the University of Wisconsin Hospital Pharmacy and paclitaxel from LC Laboratories (Woburn, Mass.). Solvents and all other reagents were purchased form Sigma Aldrich Co. (Milwaukee, Wis.) and Spectrum (Gardena, Calif.) and used as received, unless otherwise mentioned. Small molecular and polymer chromatography was accomplished with Silicycle 60 Å SiO₂ or using a Teledyne CombiFlash Rf 4x (Lincoln, NE) equipped with an evaporative light scattering detector, or ELSD, for visualization and REDI-sep Rf high performance silica or C18 columns.

¹H- and ¹⁹F-NMR spectra were obtained on a Varian Unity-Inova 400 or Varian Unity-Inova 500 spectrometer using CDC1₃ as the solvent (unless otherwise specified) and tetramethyl silane (TMS) as the internal reference. Polymer purity was confirmed by HPLC with a Gilson 321 Pump (Middleton, Wis.) equipped with a Jordi Gel DVB 500 Å (Bellingham, MA) column and a Gilson Prep-ELS detector and by MALDI-MS on a Bruker Ultraflex III MALDI TOF/TOF using α-cyano-4-hydroxycinnamic acid (CHCA) matrix unless otherwise specified.

Critical Micelle Concentration (CMC)—Surface Tensiometry

Polymer was dissolved in Millipore Milli-Q water in a 20 mL disposable scintillation vial to the desired maximum concentrations (−3 and −3.5 logM). The solutions were shaken and sonicated for 3 hours. Solutions were then allowed to equilibrate for 24 h. Serial dilutions were then prepared from these two stock solutions to achieve the desired concentrations. Each serial dilution was also sonicated 3 hours and allowed to equilibrate prior to the next dilution. Once all solutions were prepared, they were allowed to equilibrate for an additional 24 hours. Surface tensions were measured on a KSV sigma 701 tensiometer (KSV Instruments, Helsinki, Finland) equipped with a Julabo F12-MC circulator for constant temperature control. A custom round platinum rod, with a diameter of 1.034 mm and a wetted length of 3.248 mm from KSV Instruments (Helsinki, Finland), was initially cleaned with ethanol and dried in a Bunsen burner flame. Before running the experimental samples, the surface tension of Millipore Milli-Q water was measured as control to confirm vial and rod were fully cleaned and surface tension was within ±2 mN m-1 of the literature value, 78.2 mN m-1. The surface tension of each sample was then measured using the Wilhelmy method, beginning with the least concentrated solution and proceeding to successively more concentrated solutions. The surface tension at each concentration was measured in quadruplicate and the average recorded. The CMC value was determined from the intersection of the slope at the crossover point of two lines: the baseline of minimal surface tension and the slope where surface tension showed linear decline. Error was determined by weighted least squares analysis.

Nanoemulsion Preparation

Aqueous polymer solutions were prepared freshly (20 mM) in sterile, normal saline and sonicated at room temperature until fully dissolved. Saline was composed of 0.9% (w/w) sodium chloride.

Paclitaxel/medium chain triglycerides (MCT) solutions were prepared freshly. Two mL of paclitaxel solution (prepared at a concentration of 7.5 mg/mL in 50:50, acetonitrile:ethanol) was dissolved in MCT followed by heating and stirring until fully solubilized. All traces of acetonitrile and ethanol were removed by vacuum.

Paclitaxel/MCT solution and perfluoro-15-crown-5-ether (PFCE) were added to the polymer solution. The homogenizer and microfluidizer were first cleaned with 100% and 70% ethanol followed by 100% and 70% methanol and finally three rinses with Millipore Milli-Q water to remove all traces of any previous nanoemulsions. The prepared mixture was then homogenized with the high-speed homogenizer (Power Gen 500, Fisher Scientific, Hampton, N.H.) for 1 minute at 21,000 rpm at room temperature. The resulting crude emulsion was then further mixed with the microfluidizer (model 110 S, Microfluidics Corp., Newton, Mass.) for 1 minute under 5,000 psi with the cooling bath kept at 0° C. The final emulsion was then filtered with a 0.45 μm nylon filter and stored in a sterile, plastic centrifuge tube (Corning Inc., Corning, N.Y.) at 4° C.

Particle Size Determination Via Dynamic Light Scattering (DLS)

Long-term nanoemulsion size and stability was monitored via dynamic light scattering (NICOMP 380ZLS, Particle Sizing Systems, Santa Barbara, Calif.). The nanoemulsions were diluted at the intensity factor of 500 by adding 5 μL of the nanoemulsion to 3.0 mL of Millipore Milli-Q water. Each particle size analysis was run for 5 minutes at room temperature in a quartz cuvette and repeated three times. The data was analyzed using Gaussian analysis and reported as volume weighted average diameters.

Nanoemulsion distribution and histogram data were determined via dynamic light scattering (Zetasizer Nano-ZS, Malvern Instruments, Worchestershire, UK). The nanoemulsions were diluted at the same ratio as above, 5 μL nanoemulsion to 3.0 ML of Millipore Milli-Q water. Each particle analysis was run as a set of 10 scans in a semi-micro polystyrene cuvette at room temperature and repeated three times. The data were analyzed using Malvern software analysis and reported as volume weighted average diameters.

In Vitro Drug Release

The nanoemulsion was initially diluted by a factor of 20 (0.125 mL nanoemulsion plus 2.375 mL Millipore Milli-Q water). A time zero time point was established by diluting 100 μL diluted nanoemulsion mixture above in 900 μL acetonitrile (ACN). A 3 mL capacity SLIDE-A-LYZER Dialysis cassette (G2 2,000 MWCO from Thermo Fisher Scientific Inc., Fitchburg, Wis.) was hydrated by stirring for 12 hours in a 3 L PBS bath (300 mL 10x PBS and 2,700 mL Millipore Milli-Q water) at 37 C. After this time, the remaining diluted nanoemulsion solution (2.40 mL) was added to the cassette which was then returned to the PBS bath and allowed to stir for 1 week at 37° C.; this was performed in triplicate. Time points were taken at 0.5, 2, 3, 6, 9, 12, 24, 36, 48, 72, 96, 120, 144, and 168 hours. At each time point a long-stemmed glass pipette was used to mix the contents of the cassette three times. Then a 100 μL aliquot of nanoemulsion was removed from the cassette and diluted with 900 μL ACN. The nanoemulsion aliquot was then replaced in the cassette by 100 μL of fresh PBS solution (1×PBS). Sink conditions were maintained by replacing the 3 L PBS baths at the 3, 6, 9, and 12 hour time points and every 12 hours following.

The paclitaxel concentration remaining in the nanoemulsion was quantified by reverse phase HPLC. The HPLC system used was a Shimadzu PROMINENCE HPLC system (Shimadzu, Japan) equipped with an LC-20AT pump, SIL-20 AC HT autosampler, CTO-20 AC column over, and an SPD-M20A diode array detector. For each time point sample, 20 μL was injected into a C18 column (Agilent XDB-C18, 4.6 Å×150 mm) and eluted with an isocratic mixture of 25% water and 75% ACN. The run time was 7 minutes, the flow rate was 1.0 mL min-1, and the detection was set at 227 nm. Paclitaxel eluted at 4.15 minutes. Concentration of paclitaxel was determined by integrating the area of the peak and extrapolation from a standard calibration curve (500, 100, 50, 25, 10, 5, 2.5 μg mL-1).

In Vitro Cytotoxicity

A549, a human non-small cell lung carcinoma cell line, was used to perform three day cytotoxicity studies. A549 cells were cultured in RPMI-1640 medium containing 10% FBS. The cells were plated in 96-well plates, 5,000 cells/well, and incubated at 37° C. for 24 hours. After incubation, the cells were treated with 10 μL of each solution: emulsion solutions containing paclitaxel (0.04, 0.4, 4, 400, 4,000, 40,000 nM in relation to paclitaxel concentration), standard paclitaxel solutions (0.04, 0.4, 4, 40, 400, 4,000 nM in relation to paclitaxel concentration), and emulsion solutions with no paclitaxel (17 nM, 1,700 nM, 17 μM in relation to polymer concentration) diluted in 90 μL of fresh media and allowed to incubate for 24 hours at 37 C. On the third day, all liquid was removed from each well and 100 μL of diluted CellTiter-Blue® reagent (CellTiter-Blue® cell viability assay, Promega) was added to each well. The cells were incubated for another three hours at 37° C. The fluorescence intensity at 560 nm was analyzed using a plate reader. The cell viability in each well was calculated relative to the untreated control wells and each type of well was averaged (n=6).

In Vivo Developmental Toxicity Study

Due to the novel nature of the M2F8H18 amphiphile, there is no preceding toxicity data. Though some predictions can be made from previous work in the Mecozzi lab, a thorough study of developmental effects related to M2F8H18 was performed using an embryo-larval zebrafish model. This animal model was selected because zebrafish eggs remain transparent from fertilization until the tissues become dense as a mature adult. Several developmental endpoints can be simultaneously monitored providing valuable developmental toxicity data for new chemicals. Following short-term exposure, survival, and hatching, non-lethal malformations were monitored including curved body axis and pericardia edemas. Mortality rate was also monitored.

Zebrafish (Danio rerio) of the AB strain were obtained from our collaborator, Dr. Michael Taylor at the University of Madison-Wisconsin School of Pharmacy, where the fish were cultured until sexual maturation for crossing. The fish were maintained in a light/dark cycle of 14:10 hat 28.5° C. in egg water (0.03% Instant Ocean, Blacksburg, Va., USA). Zebrafish were fed with live brine shrimps (Artemia nauplii) twice a day. Embryos were obtained from healthy adult fish with a ratio of 1:2 for female to male. Six breeding groups were placed in separate spawning aquariums, equipped with a mesh bottom to prevent the eggs from being cannibalized. Crossing was induced in the morning when the light was turned on. One hour later, eggs free of macroscopically discernable symptoms of infection and disease were collected, rinsed with egg water and transferred into Petri dishes until chemical exposure.

The embryo-larvae toxicity assay was carried out according to previous studies in the Taylor and Mecozzi laboratories. Briefly, 8 fertilized eggs of 2 hpf (hours post-fertilization) stages were placed into each well of a 24-well plate and each filled with 600 μL egg water. Six concentrations (1 mM, 333 μM, 111 μM, 37 μM, 12.3 μM, and 4.1 μM) plus two controls were plated. The plate was covered and incubated at 28.5° C. in a light/dark cycle of 14:10 throughout the 96 hpf exposure period. The observations of zebrafish development were made directly in the well using a stereomicroscope (Nikon SMZ18) every 24 hr. Endpoints including mortality, spontaneous movement, hatching success, pericardial edema, and curved body axis, were selected to monitor the effects of M2F8H18. Embryos and larvae were considered dead when no heartbeat was observed. The number of hatched embryos and a cumulative mortality tally was recorded every 24 hr from 2 hpf. The number of larvae displaying pericardial edemas or curved body axes were also recorded every 24 hr from 2 hpf. At 96 hpf, following the final observations, representative larvae were anesthetized with 0.4% tricaine mesylate solution and mounted on petri dishes using low melting point agarose. The larvae were then photographed using a high-definition color microscope camera (Nikon DS-Fi2) and finally, euthanized.

Magnetic Resonance Imaging (MRI)

Samples were prepared fully concentrated, without any dilution. The nuclear magnetic resonance (NMR) internal temperature was maintained at 25° C. The ¹⁹F relaxation parameters T₁ and T₂, of the perfluoro-15-crown-5-ether present in the nanoemulsion particles, were measured on a Varian Unity-Inova 500 MHz (11.7 T) NMR spectrometer. The T₁ was determined using an inversion recovery experiment acquired with 12 independent, quadratically spaced variable (tau) values covering a range up to 10 times the estimated T₁, 0.75 s. The T₂ was determined using a Carr-Purcell-Meiboom-Gill (CPMG) pulse sequence experiment acquired with 12 independent, quadratically spaced variable (tau) values covering a range up to 10 times the estimated T₂, 0.32 s. For T₁ and T₂ measurements: 90° pulse=17.1 μs, nt=16, spectral width (T₁)=46948.4 Hz and spectral window (T₂)=46948.4 Hz.

MR images were acquired using an Agilent 4.7 T small animal horizontal bore scanner using a home-built ¹⁹F quadrature volume coil with a 1.5″ diameter and a 3″ length. The temperature was maintained at 25° C. A nanoemulsion stock of M2F8H18 was prepared by direct dilution of the nanoemulsion to the highest phantom concentration in sterile, normal saline (0.9% (w/w) sodium chloride). Lower phantom concentrations were made as serial dilutions from the stock solution in sterile, normal saline (0.9% (w/w) sodium chloride).

Nanoemulsion formation was confirmed by DLS. Phantom nanoemulsion solutions at PFCE concentrations of 154 mM, 61 mM, 31 mM, 3 mM, and 0 mM were transferred to polystyrene micro-centrifuge tubes and ¹H images of the phantoms were acquired using a gradient echo pulse sequence with 0.19×0.19×2 mm³ spatial resolution, 48×48 mm² field of view (FOV), 8.82 ms TR, 4.43 ms TE, 20 degree flip angle, 195.3 Hz/voxel, 16 averages and 36.2 s imaging time. ¹⁹F images of the phantoms were acquired using a fast spin echo pulse sequence with a 0.25×0.25×2.0 mm³ spatial resolution, 48×48 mm² FOV, 0.500 s TR, 16.08 ms TE, echo train length of 8 echoes, 104.2 Hz/voxel, 50 averages, and 10 min 1 s imaging time. Image background threshold was adjusted to only view above 5% voxels.

Synthesis

“BnOF8OH” 1H, 1H, 10H, 10H-monobenzyl-perfluorodecanol (1). To a dry roundbottom flask was added 1H,1H,10H,10H-perfluorodecane-1,10-diol (8.5 mmol, 4 g) and anhydrous dimethylformamide (40 mL). This solution was allowed to stir under argon for 10 minutes followed by the addition of sodium hydride (8.5 mmol, 0.20 g). This mixture was sonicated under argon, at room temperature for an additional 10 minutes then benzyl bromide (8.5 mmol, 1 mL) was added dropwise. The reaction was allowed to run for 3 hours under argon and sonication. The reaction was then taken up in 5% HCl then extracted with ethyl acetate, saturated aqueous NaHCO₃ solution, and brine. The crude mixture was dried with MgSO₄, concentrated in vacuo, and purified via silica gel column chromatography. A gradient of 0-15% ethyl acetate-hexanes was used. The separation was monitored with thin layer chromatography (TLC) and KMnO₄ staining. Isolated product resulted in 1.77 grams (37.5% yield). ¹H NMR (500 MHz, CDCl₃) δ 7.35 (m, 5H), 4.68 (s, 2H), 4.10 (td, J=15.2, 13.0, 7.6 Hz, 2H), 3.94 (t, J=13.8 Hz, 2H), 1.95 (t, J=7.5 Hz, 1 H). ¹⁹F NMR (470 MHz, CDCl₃) δ −119.36 (t, J=13.7 Hz), −121.92 (m), −122.42 (t, J=13.6 Hz), −123.31 (bs), −123.57 (bs).

“H18)Ms” octadecyl methanesulfonate (2). To a dry roundbottom flask was added anhydrous dichloromethane (DCM), 100 mL, 1-octadecanol (21 mmol, 5.6808 g), and triethyl amine (51 mmol, 7.10 mL). This mixture was stirred and gently heated until all solid 1-octadodecanol was dissolved. Then the flask was put in an ice bath under argon. This mixture was allowed to react for 30 minutes before methanesulfonyl chloride (27 mmol, 2.10 mL) was added dropwise. The solution turned slightly cloudy. After running overnight, the solution was diluted with more DCM and extracted with saturated aqueous NH₄Cl solution. The crude mixture was then dried with MgSO₄ and concentrated in vacuo. Isolated product resulted in 7.15 grams (97.7% yield). ¹H NMR (400 MHz, CDCl₃) δ 4.22 (t, J=6.6 Hz, 2H), 3.00 (s, 3H), 1.75 (p, J=6.9 Hz, 2H), 1.39 (m, 3H) 1.26 (bs, 31 H), 0.88 (t, J=6.6 Hz, 3H).

“BnOF8OH18” 1H, 1H, 10H, 10H-monobenzyl-octadecyl-perfluorodecane (3). To a dry roundbottom flask was added anhydrous benzotrifluoride (50 mL) and 1 (3.4 mmol, 1.90 g). This mixture was flushed with argon before adding sodium hydride (14 mmol, 0.3418 g). The mixture was allowed to stir for one hour before 2 was added (4.4 mmol, 1.5611 g). The mixture was then heated to reflux for 24 hours. After running overnight, the solution was diluted with DCM and extracted with saturated aqueous NH₄Cl solution. The crude mixture was then dried with MgSO₄ and concentrated in vacuo. Purification was performed via silica gel chromatography using a gradient of 0-5% ethyl acetate-hexanes. The separation was monitored with thin layer chromatography and KMnO₄ staining. Isolated product resulted in 2.53 grams (91.3% yield). ¹H NMR (500 MHz, CDCl₃) δ 7.35 (m, 5H), 4.68 (s, 2H), 3.93 (q, J=13.6 Hz, 4H), 3.59 (t, J=6.6 Hz, 2H), 1.60 (p, J=6.7 Hz, 2H), 1.25 (m, 32H), 0.88 (t, J=6.8 Hz, 3H). ¹⁹F NMR (470 MHz, CDCl₃) δ −119.75 (t, J=15.7 Hz), −120.03 (t, J=15.7 Hz), −121.96 (m), −123.40 (d, J=78.8 Hz).

“HOF8OH18” 1H, 1H, 10H, 10H-octadecyl-perfluorodecanol (4).

Route 1) To a dry roundbottom flash was added reagent grade methanol (100 mL), anhydrous tetrahydrofuran (50 mL), and 3 (2.90 mmol, 2.34 g). This mixture was stirred and flushed with argon for one hour then palladium on carbon catalyst (3.9 mmol, 0.4100 g) was added. This mixture was flushed with argon for one more hour then a balloon of hydrogen gas was dispelled into the flask with a bubbler attached. A second balloon of hydrogen gas was then put on the reaction and it was allowed to stir and run for 24 hours under static hydrogen atmosphere. To workup the reaction, the balloon was removed and the flask flushed with argon for one hour again. The mixture was then filtered through celite with copious rising with DCM. Solvent was removed in vacuo. Isolated product resulted in 2.25 grams (96.2% recovered yield). ¹H NMR (500 MHz, CDCl₃) δ 4.10 (td, J=14.0, 7.5 Hz, 2H), 3.92 (t, J=14.0 Hz, 2H), 3.59 (t, J=6.6 Hz, 2H), 1.93 (t, J=7.6 Hz, 1H), 1.60 (p, J=6.5 Hz, 2H), 1.25 (m, 24H), 0.88 (t, J=6.9 Hz, 3H). ¹⁹F NMR (470 MHz, CDCl₃) δ −119.65 (m), −121.96 (m), −122.42 (t, J=12.6 Hz), −123.51 (d, J=51.2 Hz).

(Route 2) To a dry round bottom flask was added 1H, 1H, 10H, 10H-perfluorodecane-1,10-diol (14.4 mmol, 6.6557 g) and anhydrous dimethylformamide (220 mL). The mixture was stirred and flushed with argon until fully dissolved then sodium hydride (14.5 mmol, 0.3482 g) was added. This was allowed to sonicate for 30 minutes, under argon before 2 (14.3 mmol, 5.0 g) was added. The reaction was allowed to run for 21 hours under argon and sonication. The reaction was then taken up in 5% HCl then extracted with ethyl acetate, saturated aqueous NaHCO₃ solution, and brine. The crude mixture was dried with MgSO₄ and concentrated in vacuo. Purification was performed using automated column chromatography, CombiFlash®, using a silica gel column. A gradient of 0-20% ethyl acetate-hexanes was used. The separation was monitored using an evaporative light scattering detector (ELSD). Isolated product resulted in 1.84 grams (17.9% yield). ¹H NMR (500 MHz, CDCl₃) δ 4.10 (td, J=15.5, 13.1, 7.6 Hz, 2H), 3.92 (t, J=13.9 Hz, 2H), 3.59 (t, J=6.6 Hz, 2H), 1.96 (t, J=7.5 Hz, 1 H), 1.60 (p, J=7.5 Hz, 2H), 1.25 (m, 33H), 0.88 (t, J=6.9 Hz, 3H). ¹⁹F NMR (470 MHz, CDCl₃) δ −119.63 (t, J=13.7 Hz), −121.95 (m), −122.43 (t, 13.5 Hz), −123.53 (d, J=46.5 Hz).

“M2OMs” monomethyl poly(ethylene glycol) methanesulfonate (5). To a dry roundbottom flask, in an ice bath and under argon, was added anhydrous dichloromethane (50 mL), poly(ethylene glycol) monomethyl ether 2,000 (2.5 mmol, 5.0195 g), and triethyl amine (7.5 mmol, 1.50 mL). This was allowed to react for 30 minutes before methanesulfonyl chloride (6.25 mmol, 0.50 mL) was added dropwise. After running overnight, the reaction was diluted with more DCM and extracted with saturated aqueous NH₄Cl solution. The crude mixture was dried with MgSO₄ and concentrated in vacuo. Purification was performed on the crude mixture dissolved in minimal DCM via precipitation of pure 5 with cold ether in a dry ice/acetone bath. Isolated product resulted in 4.93 grams (94.1% yield). ¹H NMR (400 MHz, CDCl₃) δ 4.38 (m, 2H), 3.64 (m, 182H), 3.38 (s, 3H), 3.09 (s, 3H).

M2F8H18 (6). To a dry round bottom flask was added 4 (3.7 mmol, 2.63 g) and anhydrous benzotrifluoride (150 mL). This mixture was cooled over ice, flushed with argon, and then sodium hydride was added (7.4 mmol, 0.1831 g). This reaction was stirred for 30 minutes then 5 was added (1.9 mmol, 3.4526 g). The flask was heated to reflux and allowed to stir and reflux for 5 days. Upon completion, the reaction was cooled, diluted with DCM, and extracted with saturated aqueous NH₄Cl solution. The crude mixture was dried with MgSO₄ and concentrated in vacuo. Purification was performed via automated column chromatography, CombiFlash, using a C18 column and reverse phase conditions of a 90-0% water-methanol (0.1% formic acid) to 0-100% dichloromethane-methanol gradient. Polymer purity was confirmed via NMR, HPLC, and MALDI-MS. Isolated product resulted in 2.57 grams (83.9% yield). ¹H NMR (400 MHz, CDCl₃) δ 4.04 (t, J=14.1 Hz, 2H), 3.92 (t, J=14.0 Hz, 2H), 3.79 (m, 3H), 3.64 (m, 174 H), 3.47 (t, J=5.0 Hz, 1 H), 3.38 (s, 3H), 1.60 (p, J=7.0 Hz, 3H), 1.26 (m, 36H), 0.88 (t, J=6.7 Hz, 4H). ¹⁹F NMR (376 MHz, CDCl3) δ −119.73 (dt, J=58.9, 11.9 Hz), −121.97 (m), −123.47 (bs). MALDI-MS [M+Na]⁺ calculated for C₁₁₃H₂₁₂F₁₆O₄₄Na⁺: 2600.40 m/z; found: 2600.584 m/z. [M+K]⁺ calculated for C₁₁₃H₂₁₂F₁₆O₄₄K⁺: 2616.37 m/z\; found: 2616.556 m/z.

Results and Discussion

Synthesis of M2F8H18 Semifluorinated Polymer

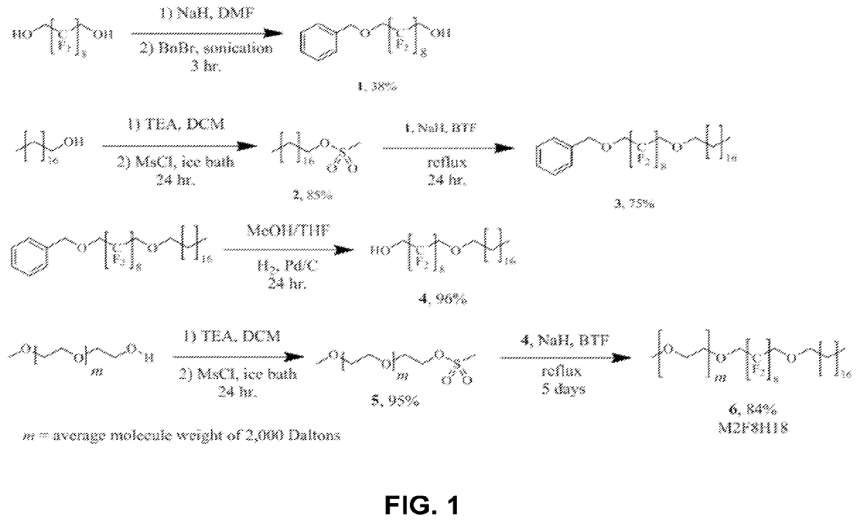

The synthesis of M2F8H18 was performed using two routes and adapted from previous work in the Mecozzi group as well as other published works. The nomenclature here will involve Mx representing poly(ethylene glycol) monomethyl ether where x is the average molecular weight in the thousands, Fy representing the fluorocarbon block where y is the number of carbons attached to fluorine atoms, and Hz representing the hydrocarbon block where z is the number of carbons attached to hydrogens. Route 1 begins with the mono-benzylation of 1H,1H,10H,10H-perfluorodecane-1,10-diol under basic conditions and sonication to yield the protected fluorous alcohol, 1. Next, 1-octadecanol was mesylated under basic conditions to isolate 2. Compounds 1 and 2 were coupled under basic conditions and reflux to the benzylated diblock intermediate, 3. Hydrogenation of 3 catalyzed by palladium on carbon resulted in the alcohol diblock intermediate, 4. Then, monomethyl poly(ethylene glycol) (mPEG) with average molecular weight of 2,000 was mesylated under basic conditions, 5. Finally, the alcohol diblock intermediate 4 was coupled to mesylated mPEG 5 under basic conditions and reflux to afford the final, desired M2F8H18 polymer, 6. Compound 6 was purified using an automated CombiFlash® system resulting in very high yields of isolated amphiphile. See FIG. 1.

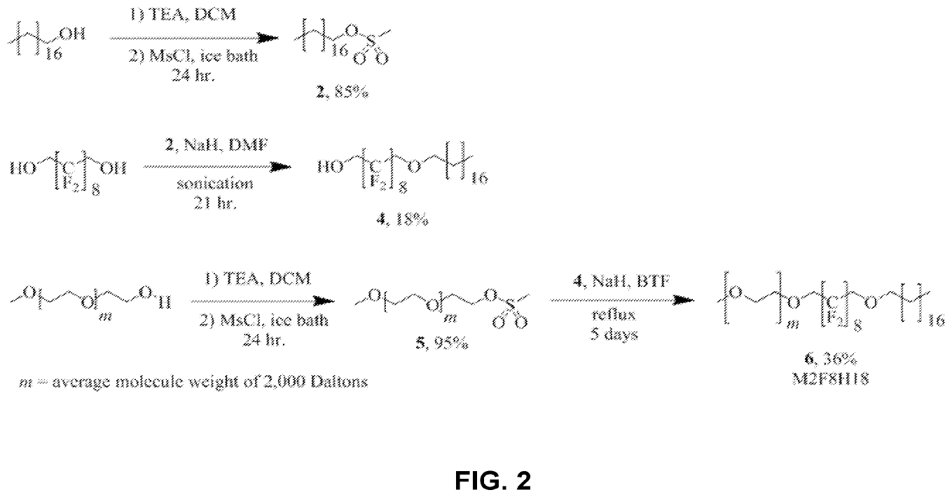

Alternatively, synthetic route 2 begins with the mesylation of 1-octadeancol under basic conditions to compound 2. Next, 1H,1H,10H,10H-perfluorodecane-1,10-diol was coupled directly to 2 without prior protection steps, as seen in Scheme 1. This coupling reaction occurred under basic conditions and sonication to directly afford 4 after purification. The final two steps mimic what was accomplished in scheme 1 including mesylation of mPEG, 5, and the final coupling of 4 and 5 under basic conditions and reflux. The final M2F8H18 amphiphile, 6, was once again purified using an automated CombiFlash® system. Resulting yields were lower in the initial reactions but overall synthesis length was reduced by two steps. See FIG. 2.

Synthetic route 1 proved to be reliable and high yielding, overall, though it does involve a more extensive set of reactions. On the other hand, synthetic route 2 significantly shorted the overall reaction scheme by two steps but with a moderate loss of yield in the initial reactions.

Physiochemical Characterization of Amphiphilic Aggregates

The Critical micelle concentration (CMC) of the M2F8H18 micellar aggregates was measured using surface tensiometry. The concentration that induces aggregation for M2F8H18, 2.2×10⁻⁶ M, is an intermediate value compared to other Mecozzi polymers M1F13 and M2H18. The standard polymer, M2DSPE (1,2-distearoyl-sn-glycero-3-phosphoethanolamine-N-[amino(polyethylene glycol)-2000], was also measured for reference comparison. The size of the micellar aggregates was also analyzed using DLS (Table 1). Based on aggregate size (Table 1) it can be concluded that M2F8H18 forms compact, stable particles.

TABLE 1
Physiochemical characteristics of aggregates.

| | | Micellar |
|---|---|---|
| Polymer | CMC (log M) | Particle Size (nm) |
| M2F8H18 | −5.7 ± 0.2 | 16.3 ± 4.5 |
| M1F13 | −6.1 ± 0.1 | 17.2 ± 1.9 |
| M2H18 | −5.1 ± 0.1 | 12.2 ± 3.1 |
| M2DSPE | −4.9 ± 0.2 | 13.9 ± 1.6 |

Nanoemulsion Preparation and Stability

Formulation of M2F8H18 nanoemulsions with MCT or an MCT/Paclitaxel (PTX) combination was very successful resulting in opaque, milky colloidal solutions ranging in size from 200-250 nm initially. High concentrations of paclitaxel, from 1-4 mg drug per mL nanoemulsion, were loaded into the core of the particles. See FIG. 3 for proposed therapeutic nanoemulsion structure.

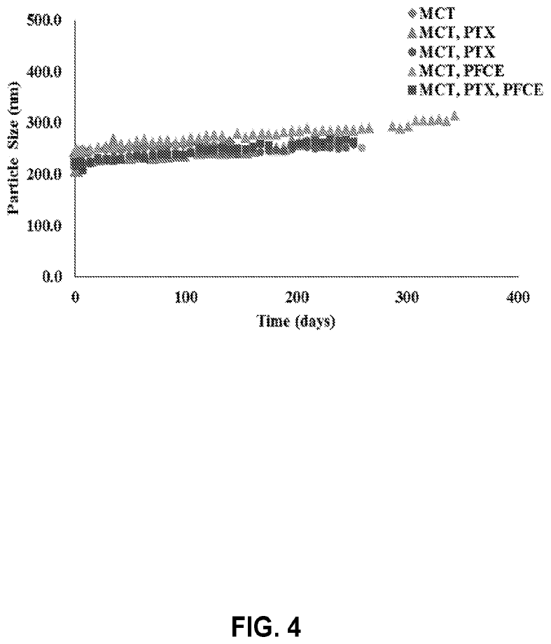

Nanoemulsions containing the highest concentrations of paclitaxel were stable for over one year, though some drug precipitation was noted over time. As seen below (FIG. 4), the M2F8H18 nanoemulsions also exhibit very minimal Ostwald ripening, in stark contrast to many of the previous nanoemulsions on the market. Some minimal particle growth can be observed over time but as a whole the colloids display steady, consistent size. The nanoemulsion sizes observed for M2F8H18 are appropriate to take advantage of the EPR effect, up to 400 nm, and significantly below the FDA standard size cutoff for nanoparticles to be delivered in vivo, 500 nm. Not only that, but the particles exhibit long-term stability required for consideration for in vivo treatment, up to one year here. The addition of the PFCE imaging agent resulted in increased colloidal stability and better drug retention, as hypothesized by the design of the M2F8H18 amphiphile. This added stability from the fluorinated excipient further supports the idea that the thicker the fluorous core surrounding the oil droplet, the more controlled the drug release will be, which is critical for the successful administration of chemotherapeutics. See FIG. 4, which shows change in particle size of M2F8H18 nanoemulsions over time as measured by DLS: (diamond) 4 mL MCT only, (triangle) 2 mL MCT and 1 mg/mL paclitaxel, (circle) 4 mL MCT and 4 mg/mL paclitaxel, (darker triangle) 2 mL MCT and 10% v/v PFCE, (square) MCT, 1 mg/mL paclitaxel, and 23.5% v/v PFCE. All nanoemulsions prepared in 0.9% w/w NaCl.

In Vitro Drug Release Profile

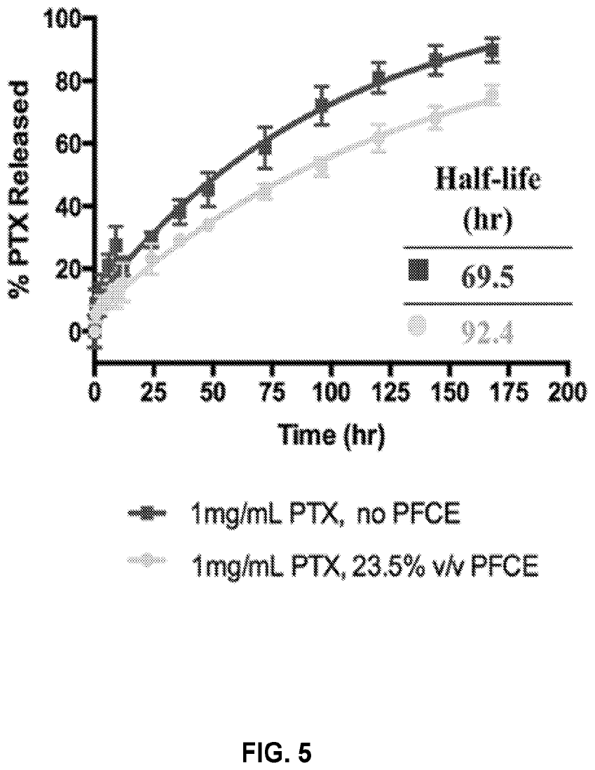

The in vitro paclitaxel release profile for M2F8H18 showed slow, steady release with highly sustained half-lives. Burst release is a common issue exhibited by many nanoparticles but no trace of that is seen here. The linear M2F8H18 alone improved greatly upon reported in vitro paclitaxel release from micellar delivery systems, to a very promising 69.5 hours (FIG. 5). Micelles for delivery of paclitaxel show much faster release (see, e.g., Lu et al., International J. of Pharmaceutics, 471 (2014) 525-535; Zhang et al., J. Am. Chem. Soc. 2015, 137, 2056-2066). The time-release half-life for paclitaxel in these art-known micelles can be estimated to be about 1 hr in one case and seven hours in the other. Thus, the systems of the invention are much superior, although as discussed elsewhere herein, the nanoemulsions of the present invention are emulsions, not micelles. This delay in controlled release for the instant invention can be attributed to the penetration of the long hydrocarbon chains of M2F8H18 into the MCT oil droplet where the drug is solubilized. Additionally, the perfluorocarbon shell surrounding the oil droplet provides a barrier to modulate drug release from the oil droplet as well as provide enhanced stability to the particles. Upon addition of the contrast agent, PFCE, in the theranostic formulation below (FIG. 5) the drug release is further modulated to an amazing half-life of 92.4 hours. Not only does the PFCE add imaging potential to the nanoparticles but it also increases the size of the fluorous phase and aggregates around the F8 shell of the polymer chains. This provides enhanced driving force toward particle formulation as well as long-term stability. The higher concentration of PFCE that is added, the thicker the fluorous shell around the oil droplet, therefore greatly slowing paclitaxel release.

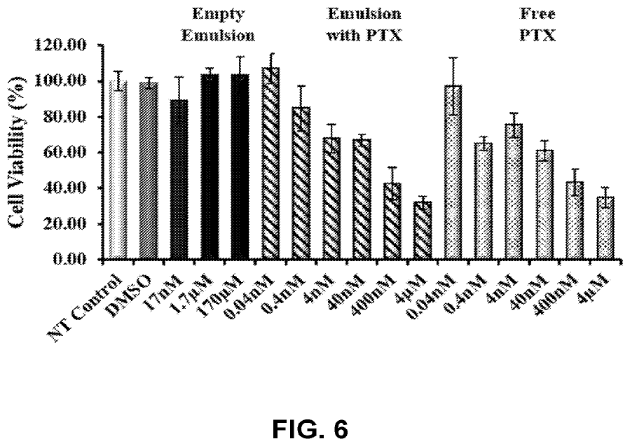

Cellular cytotoxicity was examined in vitro using A549 non-small cell lung cancer cells. The A549 cells were selected due to their robust nature and frequent use in the field. Following cell plating and incubation, the cells were treated with three solutions comprising of 10 μL of each solution, diluted with 90 μL of fresh media. Solutions included a M2F8H18 nanoemulsion containing 4 mg/mL paclitaxel with 4 mL MCT, a M2F8H18 nanoemulsion containing no paclitaxel with 4 mL MCT, and paclitaxel standard solutions prepared with low concentrations of dimethyl sulfoxide (DMSO). Because DMSO has been shown to be toxic to A549 cells at high concentrations DMSO cytotoxicity was also monitored in this study. Paclitaxel standard concentrations were prepared at least three magnitudes above and two below its reported IC₅₀ for the A549 cell line, 4 nM. Following treatment and incubation, the cell viability was measured using a CellTiter-Blue® cell assay. The fluorescence intensity of each well was measured by a plate reader, at 560 nm, and cell viability was then calculated in relativity to the untreated control wells. All wells treated with the same solution were averaged, n=6. As seen below (FIG. 6), the media containing 0.05% DMSO, which is the highest concentration used in this study, showed little to no cell inhibition confirming that it was appropriate to use low concentrations of DMSO in solution preparation without any cytotoxic effects. The wells treated with the M2F8H18 nanoemulsion void of drug exhibited very high cell viability, confirming that this novel polymer and MCT oil do not inhibit cell growth and are safe for in vivo use. The M2F8H18 nanoemulsion loaded with high concentrations of paclitaxel did inhibit cell growth at similar concentrations as the standard paclitaxel solutions. In fact, the paclitaxel delivered via the nanoemulsion inhibited cell growth at an even lower concentration compared to the standards. These data suggest that the chemotherapeutic containing nanoemulsions can successfully inhibit cancer cell growth, as they were designed to do. At this point, the mode of delivery is unclear and currently being probed in other studies. FIG. 6 shows A549 cell viability studies: (far left) non-treated control, (second left) 0.05% DMSO control, (3^{rd} through 5^{th} columns) M2F8H18 nanoemulsion with no drug and MCT, (6^{th} through 11^{th} columns) M2F8H18 nanoemulsion loaded with 4 mg/mL paclitaxel and MCT, (12^{th} through 17^{th} columns) paclitaxel standard solutions.

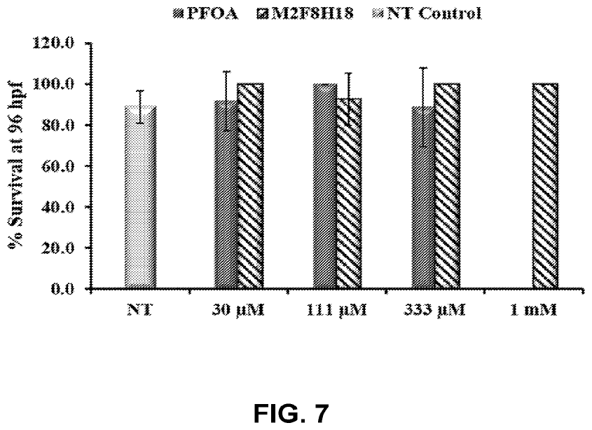

The M2F8H18 amphiphile was also screened for initial in vivo developmental toxicity, due to the novel nature of the polymer, using a zebrafish model. Though some toxicity predictions can be made from previous work in the Mecozzi lab, a thorough study of developmental effects related to M2F8H18 was performed using an embryo-larval zebrafish model. This animal model was selected because zebrafish eggs remain transparent from fertilization until the tissues become dense as a mature adult. Several developmental endpoints can be simultaneously monitored providing valuable developmental toxicity data for new chemicals. After crossing the fish and fertilization of the eggs had occurred, the embryos were collected and plated. The embryos were then treated with M2F8H18 solutions of the following concentrations: 1 mM, 333 μM, 111 μM, 37 μM, 12.3 μM, and 4.1 μM. The embryos were then incubated, monitored, and photographed over a 96-hour experiment period. Survival rates for the non-treated control fish (FIG. 7) were very high, as would be expected from healthy zebrafish at 96 hpf. The positive control for the experiment, perfluorooctanoic acid, was selected for its documented toxicity to zebrafish and the contrasting difference between perfluoro acids and Mecozzi polymers. The PFOA caused mortality in all fish at the highest concentration of 1 mM as well as 67% of fish at the next highest concentration of 333 μM. Select fish at 333 μM also exhibited a curved body axis and mild pericardia edemas. The results are varied at concentrations at and lower than 111 μM indicating sub-lethal concentrations may have been reached. No other malformations were noted at the lower PFOA concentrations. In great contrast, the novel M2F8H18 polymer induced no mortalities in the fish of this study, even at the highest concentration of 1 mM. The small decrease in survival rate, to 78%, seen at 111 μM is thought to be a random anomaly based on the collective zebrafish data. None of the fish exposed to M2F8H18 in this study exhibited malformations such as curved body axis or pericardia edemas, as well. These data suggest that there are no developmental toxicity concerns involving 1mM and below concentrations of the newly synthesized M2F8H18 polymer. Research in this area may progress to further in vivo stages. FIG. 7 shows zebrafish survival rates after exposure for 96 hpf: (for left) non-treated control, (middle) positive control of perfluoro-octanoic acid, (right) M2F8H18 polymer.

¹⁹F-MR Imaging Characterization

It has now been established that the M2F8H18 nanoemulsions can be loaded with high concentrations of PFCE. The addition of the excipient fluorocarbon enhances the drug release profile of the nanoemulsions and provides further stability to the colloidal particles. Next, the imaging potential of this molecule was investigated. Preliminary ¹⁹F-NMR studies at 11.7 T show an intense, single peak at 6 -91.8 ppm with CFCl₃ as an internal refernce.15 This pattern is due to the twenty magnetically equivalent fluorine atoms and ring flexibility in solution of the PFCE. The ¹⁹F relaxation parameters T1, longitudinal relaxation or delay between pulses, and T2, transverse relaxation or signal decay over time, were also measured to monitor potential PFCE use as an efficient contrast agent. Nanoemulsion samples were prepared fully concentrated, without any dilution. The T1 was determined using an inversion recovery experiment while T2 was determined using a Carr-Purcell-Meiboom-Gill (CPMG) pulse sequence experiment. The resulting relaxation values (Table 1) show a relatively short T1 value allowing for quick recovery between pulses and increased scanning efficiency as well as a long T2 value that should avoid most signal decay over time. Based on the single resonance intensity, high payload concentration within the nanoemulsions, and good relaxation values the PFCE has potential for use as a 19F-MR contrast agent.

TABLE 2

| Fluorous Moiety | | |
|---|---|---|
| Measured | T₁ (sec) | T₂ (sec) |
| M2F8H18/PFCE | 0.781 ± 0.002 | 0.163 ± 0.006 |
| nanoemulsion | | |

¹⁹F relaxivity measurements for perfluoro-15-crown-5-ether at 11.7 T. PFCE exhibits favorable 19F-NMR characteristics amenable for translation to ¹⁹F-MR imaging: a single 19F resonance, a relatively small T₁ value, and a relatively large T₂ value.

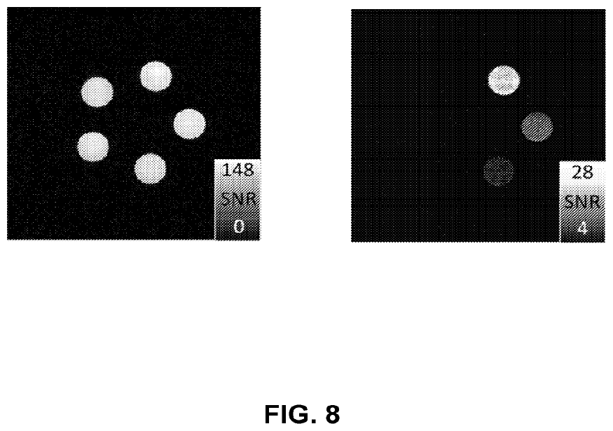

Following preliminary work to establish the relaxation parameters of PFCE, ¹⁹F-MR phantom images of the PFCE loaded nanoemulsions were acquired using a 4.7 T small animal MRI instrument. The ¹H images (FIG. 8) were acquired over an imaging time of 36.2 seconds while the ¹⁹F images over an imaging time of 10 minutes and 1 second. The nanoemulsion exhibited very high signal intensity from primarily the saline solution, in the ¹H images, and the PFCE, in the ¹⁹F images. The MRI probe had no issue tuning and shimming to both the ¹H and ¹⁹F frequencies. ¹⁹F-MR signal intensity is extremely high at the fully concentrated nanoemulsion sample (top-center, FIG. 8) with PFCE concentration of 154 mM. Excellent signal intensity can also be seen at the 31 mM concentration with the limit of detection falling somewhere below 31 mM and above 3 mM. Image signal to noise ratio could possibly be amplified with a different pulse sequence or an instrument of higher field strength. These data demonstrate the great potential for use of these theranostic nanoemulsions in vivo. Future work will attempt imaging with the M2F8H18/PFCE nanoemulsion in a tumor mouse model. FIG. 8 shows ¹H-MR (left) and ¹⁹F-MR (right) phantom images taken at 4.7 T. Image background threshold was adjusted to only view above 5% voxels. From top-center moving clockwise, the concentration of PFCE in the M2F8H18 nanoemulsion is 154 mM, 61 mM, 31 mM, 3 mM, and 0 mM. Strong signal intensity can be viewed at 31 mM while 3 mM is below the limit of detection.

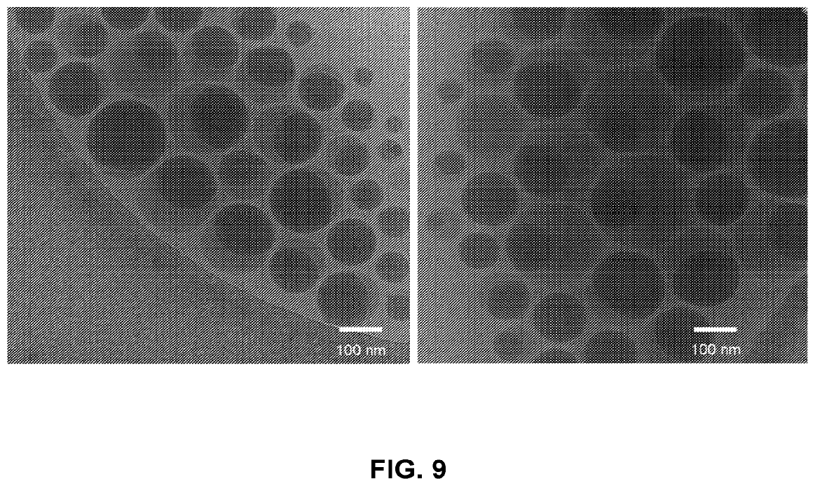

FIG. 9 shows cryogenic-electron microscopy (Cryo-EM) images of the nanoemulsions. The dark oil in the core is the perfluoro-15-crown-5-ether; the light oil in the core is the medium chain triglycerides. The images show the presence of the biphasic core, that is, a core in the nanoemulsion particles composed of a droplet of perfluorocrown ether (the imaging agent) and the droplet of hydrophobic oil in which the therapeutic agent is dissolved. The biphasic cores observed are very rare in nanoemulsions and their presence explains the ability of the materials to encapsulate such large amounts of imaging agent.

Conclusions

The novel, semifluorinated amphiphile M2F8H18 was synthesized and thoroughly characterized. No developmental toxicity was found for this polymer after initial in vivo studies in zebrafish. This redesigned ABC polymer forms incredibly stable, triphilic nanoemulsions, due to the intrinsic driving force of the fluorous phase, with life-times up to 1 year. Very limited Ostwald ripening is observed for these particles thus they remain well below 400 nm for their full life-time. The M2F8H18 surfactant can form therapeutic nanoemulsions where it stabilizes large MCT oil droplets containing solid, highly hydrophobic paclitaxel at concentrations up to 4 mg/mL. Drug release from the oil core of the particles is gradual and controlled due to the long, stabilizing hydrocarbon chains that penetrate the oil droplet and the intermediary fluorocarbon shell that acts as a barrier. The addition of the fluorous excipient PFCE modulates drug release even further by sealing the fluorous shell of the polymer. Upon addition of the PFCE, these nanoemulsions can be formulated into theranostic systems with dual therapeutic and diagnostic character. High concentrations of the PFCE can be loaded into the nanoemulsions resulting in strong ¹⁹F-MR signal due to its intense, single resonance composed of twenty magnetically equivalent fluorine atoms, short T₁, and long T₂. This preliminary work with M2F8H18 showed the polymer can formulate stable nanoemulsions that carry payloads of potent chemotherapeutic drug and ¹⁹F-MR contrast agent all wrapped into a powerful, theranostic system. Future studies will focus on the translation of these M2F8H18 therapeutic and theranostic nanoemulsions to in vivo models.

### References.

van Houtem, M. H. C. J.; Benaskar, F.; Fitié, C. F. C.; Martin-Rapún, R.; Vekemans, J. a. J. M.; Meijer, E. W. Helical Self-Assembly and Co-Assembly of Fluorinated, Preorganized Discotics. Org. Biomol. Chem. 2012, 10 (30), 5898.

CRC Handbook of Chemistry and Physics, 85th ed.; Lide, D. R., Ed.; CRC Press LLC, 2004.

Parlato, M. C.; Jee, J.-P.; Teshite, M.; Mecozzi, S. Synthesis, Characterization, and Applications of Hemifluorinated Dibranched Amphiphiles. J. Org. Chem. 2011, 76, 6584-6591.

Colloids in Drug Delivery, 1st ed.; Fanun, M., Ed.; CRC Press, Taylor & Francis Group: Boca Raton, Fla., 2010.

Maeda, H.; Nakamura, H.; Fang, J. The EPR Effect for Macromolecular Drug Delivery to Solid Tumors: Improvement of Tumor Uptake, Lowering of Systemic Toxicity, and Distinct Tumor Imaging in Vivo. Adv. Drug Deliv. Rev. 2013, 65 (1), 71-79.

Maeda, H. The Enhanced Permeability and Retention (EPR) Effect in Tumor Vasculature: The Key Role of Tumor-Selective Macromolecular Drug Targeting. Adv. Enzyme Regul. 2001, 41 (0), 189-207.

Iyer, A. K.; Khaled, G.; Fang, J.; Maeda, H. Exploiting the Enhanced Permeability and Retention Effect for Tumor Targeting. Drug Discov. Today 2006, 11 (17-18), 812-818.

Jordan, M. A.; Wilson, L. Microtubules as a Target for Anticancer Drugs. Nat. Rev. Cancer 2004, 4 (4), 253-265.

Nogales, E.; Wolf, S. G.; Khan, I. A.; Ludueña, R. F.; Downing, K. H. Structure of Tubulin at 6.5 A and Location of the Taxol-Binding Site. Nature. 1995, pp 424-427.

Singh, S.; Dash, A. K. Paclitaxel in Cancer Treatment: Perspectives and Prospects of Its Delivery Challenges. Crit. Rev. Ther. Drug Carrier Syst. 2009, 26 (4), 333-372.

Miele, E.; Spinelli, G. P.; Miele, E.; Tomao, F.; Tomao, S. Albumin-Bound Formulation of Paclitaxel (Abraxane® ABI-007) in the Treatment of Breast Cancer. Int. J. Nanomedicine 2009, 4 (1), 99-105.

Bae, Y. H.; Park, K. Targeted Drug Delivery to Tumors: Myths, Reality and Possibility. J. Control. Release 2011, 153 (3), 198-205.

Park, K. Facing the Truth about Nanotechnology in Drug Delivery. ACS Nano 2013, 7 (9), 7442-7447.

Chen, X.; Gambhir, S. S.; Cheon, J. Guest Editorial- Theranostic Nanomedicine. Acc. Chem. Res. 2011, 44 (10), 841.

Lin, T.-Y.; Lin, W.-H.; Clark, W. D.; Lagow, R. J.; Larson, S. B.; Simonsen, S. H.; Lynch, V. M.; Brodbelt, J. S.; Maleknia, S. D.; Liou, C.-C. Synthesis and Chemistry of Perfluoro Macrocycles. J. Am. Chem. Soc. 1994, 116 (m), 5172-5179.

Tran, T. D.; Caruthers, S. D.; Hughes, M.; Marsh, J. N.; Cyrus, T.; Winter, P. M.; Neubauer, A. M.; Wickline, S. A.; Lanza, G. M. Clinical Applications of Perfluorocarbon Nanoparticles for Molecular Imaging and Targeted Therapeutics. Int. J. Nanomedicine 2007, 2 (4), 515-526.

Ahrens, E. T.; Flores, R.; Xu, H.; Morel, P. A. In Vivo Imaging Platform for Tracking Immunotherapeutic Cells. Nat. Biotechnol. 2005, 23 (8), 983-987.

Srinivas, M.; Cruz, L. J.; Bonetto, F.; Heerschap, A.; Figdor, C.G.; de Vries, I. J. M. Customizable, Multi-Functional Fluorocarbon Nanoparticles for Quantitative in Vivo Imaging Using 19F MRI and Optical Imaging. Biomaterials 2010, 31 (27), 7070-7077.

Ruiz-Cabello, J.; Walczak, P.; Kedziorek, D. A.; Chacko, V. P.; Schmieder, A. H.; Wickline, S. A.; Lanza, G. M.; Bulte, J. W. M. In Vivo “hot spot” MR Imaging of Neural Stem Cells Using Fluorinated Nanoparticles. Magn. Reson. Med. 2008, 60 (6), 1506-1511.

Lanza, G. M.; Winter, P. M.; Caruthers, S. D.; Hughes, M. S.; Hu, G.; Schmieder, A. H.; Wickline, S. A. Theragnostics for Tumor and Plaque Angiogenesis with Perfluorocarbon Nanoemulsions. Angiogenesis 2010, 13 (2), 189-202.

Flögel, U.; Ding, Z.; Hardung, H.; Jander, S.; Reichmann, G.; Jacoby, C.; Schubert, R.; Schrader, J. In Vivo Monitoring of Inflammation after Cardiac and Cerebral Ischemia by Fluorine Magnetic Resonance Imaging. Circulation 2008, 118 (2), 140-148.

Idée, J. M.; Port, M.; Medina, C.; Lancelot, E.; Fayoux, E.; Ballet, S.; Corot, C. Possible Involvement of Gadolinium Chelates in the Pathophysiology of Nephrogenic Systemic Fibrosis: A Critical Review. Toxicology 2008, 248 (2-3), 77-88.

Schweighardt, F. K.; Rubertone, J. A. Perfluoro-Crown Ethers in Fluorine Magnetic Resonance Spectroscopy. U.S. Pat. No. 5, 068, 098, 1991.

Janjic, J.; Ahrens, E. Fluorine Containing Nanoemulsions for MRI Cell Tracking. Wiley lnterdiscip. Rev. . . . 2009, 492-501.

Kadayakkara, D. K. K.; Janjic, J. M.; Pusateri, L. K.; Young, W. Bin; Ahrens, E. T. In Vivo Observation of Intracellular Oximetry in Perfluorocarbon-Labeled Glioma Cells and Chemotherapeutic Response in the CNS Using Fluorine-19 MRI. Magn. Reson. Med. 2010, 64 (5), 1252-1259.

Slaughter, J. N.; Schmidt, K. M.; Byram, J. L.; Mecozzi, S. Synthesis and Self-Assembly Properties of a Novel [Poly(ethylene Glycol)]-Fluorocarbon-Phospholipid Triblock Copolymer. Tetrahedron Lett. 2007, 48 (22), 3879-3882.

Hussein, W. M.; Ross, B. P.; Landsberg, M. J.; Lévy, D.; Hankamer, B.; McGeary, R. P. Synthesis of Nickel-Chelating Fluorinated Lipids for Protein Monolayer Crystallizations. J. Org. Chem. 2009, 74 (4), 1473-1479.

Tucker, W. B. Design, Synthesis, and Physicochemical Characterization of Semifluorinated Triphilic Surfactants with Applications for Hydrophobic Drug Delivery. Ph.D. Dissertation, University of Wisconsin-Madison, Madison, Wis 2015 University of Wisconsin-Madison, 2015.

Yuan, F.; Dellian, M.; Fukumura, D.; Leunig, M.; Berk, D. A.; Torchilin, V. P.; Jain, R. K. Vascular Permeability in a Human Tumor Xenograft: Molecular Size Dependence and Cutoff Size. Cancer Res. 1995, 55, 3752-3757.

FDA. Guidance for Industry: Q1A(R2) Stability Testing of New Drug Substances and Products, U.S. Department of Health and Human Services, Food and Drug Administration; 2003.

Lu, J.; Chuan, X.; Zhang, H.; Dai, W.; Wang, X.; Wang, X.; Zhang, Q. Free Paclitaxel Loaded PEGylated-Paclitaxel Nanoparticles: Preparation and Comparison with Other Paclitaxel Systems in Vitro and in Vivo. Int. J. Pharm. 2014, 471 (1-2), 525-535.

Huh, K. M.; Lee, S. C.; Cho, Y. W.; Lee, J.; Jeong, J. H.; Park, K. Hydrotropic Polymer Micelle System for Delivery of Paclitaxel. J. Controlled Release 2005, 101 (1-3), 59-68.

Shin, H. C.; Alani, A. W. G.; Cho, H.; Bae, Y.; Kolesar, J. M.; Kwon, G. S. A 3-in-1 Polymeric Micelle Nanocontainer for Poorly Water-Soluble Drugs. Mol. Pharmaceutics 2011, 8 (4), 1257-1265.

Kreja, L.; Seidel, H. J. On the Cytotoxicity of Some Microbial Volatile Organic Compounds as Studied in the Human Lung Cell Line A549. Chemosphere 2002, 49 (1), 105-110.

Liebmann, J. E.; Cook, J. A; Lipschultz, C.; Teague, D.; Fisher, J.; Mitchell, J. B. Cytotoxic Studies of Paclitaxel (Taxol) in Human Tumour Cell Lines. Br. J. Cancer 1993, 68 (6), 1104-1109.

Hagenaars, A.; Vergauwen, L.; Benoot, D.; Laukens, K.; Knapen, D. Mechanistic Toxicity Study of Perfluorooctanoic Acid in Zebrafish Suggests Mitochondrial Dysfunction to Play a Key Role in PFOA Toxicity. Chemosphere 2013, 91 (6), 844-856.

Hagenaars, A.; Vergauwen, L.; De Coen, W.; Knapen, D. Structure-Activity Relationship Assessment of Four Perfluorinated Chemicals Using a Prolonged Zebrafish Early Life Stage Test. Chemosphere 2011, 82 (5), 764-772.

Ruiz-Cabello, J.; Barnett, B. P.; Bottomley, P. A.; Bulte, J. W. M. Fluorine (19F) MRS and MRI in Biomedicine. NMR Biomed. 2011, 24 (2), 114-129.

### STATEMENTS REGARDING INCORPORATION BY REFERENCE AND VARIATIONS

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomer and enantiomer of the compound described individually or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous with “including, ” “containing, ” or “characterized by, ” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. An emulsion for delivery of a therapeutic agent and/or an imaging agent, said emulsion comprising an oil in water emulsion comprising:
a hydrophobic liquid;
an aqueous solution;
semi-fluorinated block copolymers; wherein each of said semi-fluorinated block copolymers independently comprises a hydrophilic block, a hydrophobic block and a fluorophilic block; wherein said fluorophilic block of each of said semi-fluorinated block copolymers is provided between said hydrophobic block and said hydrophilic block;
wherein said hydrophobic liquid is capable of solubilizing the therapeutic agent; and
wherein said fluorinated block is capable of solubilizing the imaging agent.

2. The emulsion of claim 1, wherein said hydrophobic liquid is lipophilic.

3. The emulsion of claim 1, wherein said hydrophobic liquid is one or more oils.

4. The emulsion of claim 3, wherein said hydrophobic liquid is one or more glycerides.

5. The emulsion of claim 3, wherein said hydrophobic liquid is one or more medium-chain triglycerides.

6. The emulsion of any of claims 1-5, wherein said semi-fluorinated block copolymers have a concentration selected from the range of 5 mg L⁻¹ to 50 mg L⁻¹, or a concentration selected from the range of 5 to 30 mM.

7. The emulsion of any of claims 1-6, wherein said hydrophobic block of each of said semi-fluorinated block copolymers is a polymer terminating group.

8. The emulsion of any of claims 1-7, wherein said hydrophilic block of each of said semi-fluorinated block copolymers is a polymer terminating group.

9. The emulsion of any of claims 1-8, wherein said hydrophobic block of each of said semi-fluorinated block copolymers is directly linked to said fluorophilic block.

10. The emulsion of any of claims 1-9, wherein said fluorophilicblock of each of said semi-fluorinated block copolymers is directly linked to said hydrophilic block.

11. The emulsion of any of claims 1-10, wherein said hydrophobic block, said hydrophilic block or both are independently linked to said fluorophilic block via a linking moiety selected from the group consisting of an ether group, a carbamate group, an amide group, a carboxyl group, an ester group, an alkyl group, an alkylene group, an amino group or any combination of these.

12. The emulsion of any of claims 1-11, wherein each of said fluorophilic blocks of said semi-fluorinated block copolymers is independently a fluorocarbon moiety having between 3 to 32 carbon-fluorine bonds.

13. The emulsion of any of claims 1-12, wherein each of said fluorophilic blocks of said semi-fluorinated block copolymers is independently a fluorinated alkyl group having a length of 6 to 16 carbons.

14. The emulsion of any of claims 1-13, wherein each of said fluorophilic blocks of said semi-fluorinated block copolymers is independently a perfluorinated alkyl group having a length of 6 to 16 carbons.

15. The emulsion of any of claims 1-14, wherein said hydrophilic blocks of said semi-fluorinated block copolymers is independently selected from the group consisting of a polyoxygenated polymer block, a poly(vinylpyrrolidone) block, a poly(acrylic) block, a polyacrylamide block, a polyoxazoline block, a polysaccharide block and a chitosan derivative block.

16. The emulsion of any of claims 1-15, wherein each of said hydrophilic blocks of said semi-fluorinated block copolymers is a poly(ethylene glycol) block having an average molecular weight selected over the range of 1000 g mol⁻¹ to 40, 000 g mol⁻¹.

17. The emulsion of any of claims 1-16, wherein each said hydrophobic blocks of said semi-fluorinated block copolymers is independently selected from the group consisting of a substituted or unsubstituted C₅-C₂₇ alkyl block, substituted or unsubstituted C₅-C₂₇ alkylene block, a poly (ϵ-caprolactone) block, a poly(lactic acid) block; a poly(propylene glycol) block; a poly(amino acid) block; a poly(ester) block and poly(lactic-co-glycolic acid), wherein the block may be linear or branched.

18. The emulsion of any of claims 1-17, wherein each of said hydrophobic blocks of said semi-fluorinated block copolymers is independently an unsubstituted C₁₂-C₂₀ alkyl group.

19. The emulsion of any of claims 1-18, wherein each of said hydrophobic blocks of said semi-fluorinated block copolymers is independently a C₁₆ alkyl group.

20. The emulsion of any of claims 1-19, wherein each of said semi-fluorinated block copolymers independently has the formula (FX1): 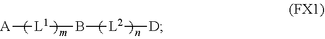
wherein A is the hydrophilic block, B is the fluorophilic block and D is the hydrophobic block;
wherein L¹ and L² are each independently a linking group; and wherein m is 0 or 1 and n is 0 or 1.

21. The emulsion of claim 20, wherein each of said semi-fluorinated block copolymers independently has the formula (FX2): 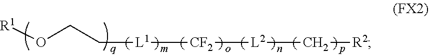
wherein q is a integer selected from the range of 10 to 300, o is an integer selected from the range of 6 to 16, and p is an integer selected from the range of 10 to 27;
wherein R¹ is hydrogen, methyl, C₁-C₁₀ alkyl, C₃-C₁₀ cycloalkyl, 0₅-C₁₀ aryl, C₅-C₁₀ heteroaryl, C₁-C₁₀ alkoxy or C₁-C₁₀ acyl;
wherein R² is hydrogen or C₁-C₅ alkyl;
wherein each of L¹ and L² is independently null, —O—, —O(CH₂)ₑ—, —(CH₂)ₑ—, —(CH₂)ₑO(CH₂)_{f}—, —(CH₂)ₑS(CH₂)_{f}—, —(CH₂)ₑNR¹¹(CH₂)_{f}—, —(CH₂)ₑOCONR¹²(CH₂)_{f}—, —(CH₂)ₑCONR¹³(CH₂)_{f}—, —(CH₂)ₑNR¹⁴COO(CH₂)_{f}—, —(CH₂)ₑNR¹⁵CO(CH₂)_{f}— or —(CH₂)ₑNR¹⁶CONR¹⁷(CH₂)_{f}—;
wherein each of R¹¹-R¹⁷ is independently hydrogen, methyl, or C₁-C₅ alkyl; and
wherein each of e and f is independently an integer selected from the range of 0 to 5.

22. The emulsion of claim 21, wherein each of said semi-fluorinated block copolymers independently has the formula (FX3A) or (FX3B):

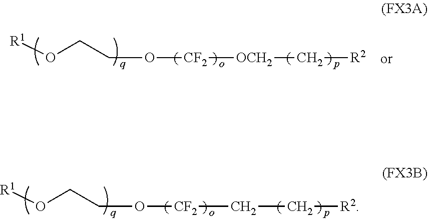

23. The emulsion of claim 21, wherein each of said semi-fluorinated block copolymers independently has the formula (FX4A) or (FX4B):

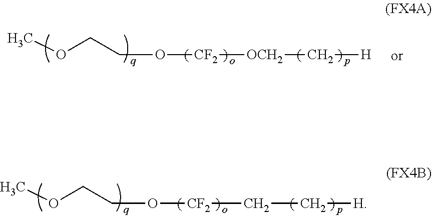

24. The emulsion of any of claims 1-23, wherein each of said semi-fluorinated block copolymers independently has a molecular weight selected from the range 1100 Da to 14000 Da.

25. The emulsion of any of claims 1-24, further comprising a perhalogenated fluorous compound.

26. The emulsion of claim 25, wherein said perhalogenated fluorous compound is 3% to 40% by volume of said emulsion.

27. The emulsion of claim 25, wherein said perhalogenated fluorous compound is selected from the group consisting of perfluorooctyl bromide, perfluorononyl bromide, perfluorodecyl bromide, perfluorodecalin, perfluorodichlorooctane, bis-perfluorobutyl ethylene and perfluoro(methyldecalin).

28. The emulsion of any of claims 1-27, further comprising a therapeutic agent.

29. The emulsion of claim 28, wherein the therapeutic agent has a concentration selected from the range of 0.1 mg mL⁻¹ to 50 mg mL⁻¹ relative to the hydrophobic liquid of said emulsion.

30. The emulsion of any of claims 28-29, wherein said therapeutic agent is a hydrophobic compound and is noncovalently associated with the hydrophobic block of said semi-fluorinated block copolymers.

31. The emulsion of any of claims 28-30, wherein said therapeutic agent is characterized by a solubility in water of equal to or less than 20 mM.

32. The emulsion of any of claims 28-31, wherein said therapeutic agent is an anticancer agent or antifungal agent.

33. The emulsion of claim 32, wherein said therapeutic agent is selected from the group consisting of paclitaxel, doxorubicin, retinoic acid series, camptothecin, docetaxel, tamoxifen, anasterozole, topotecan, belotecan, irinotecan, gleevec and vincristine.

34. The emulsion of claim 33, wherein the therapeutic agent is paclitaxel and said paclitaxel has a concentration of 0.1 mg mL⁻¹ to 50 mg mL⁻¹ relative to the hydrophobic liquid in said emulsion.

35. The emulsion of any of claims 1-34 further comprising an imaging agent.

36. The emulsion of claim 35, wherein the imaging agent is a nuclear magnetic resonance imaging contrast agent.

37. The emulsion of claim 35, wherein the imaging agent is physically associated with the fluorophilic blocks of semi-fluorinated block copolymers.

38. The emulsion of claim 36, wherein the imaging agent is a selected from the group consisting of a perfluorinated compound selected from the group consisting of perfluoroalkanes, perfluoroalkylamines, perfluoro-crown-ethers, perfluorinated alcohols, perfluorohaloalkanes, perfluorinated carboxylic acids, perfluorinated acrylates, and perfluorinated esters.

39. The emulsion of claim 38, wherein the imaging agent is perfluoropolyether, perfluoro-15-crown-5-ether, sulfur hexafluoride, hexafluoroethane, or perfluoropropane.

40. The emulsion of claim 35, wherein said imaging agent is perfluoro-15-crown-5-ether and has a concentration of either 5 mg mL⁻¹ to 750 mg mL⁻¹ or of from about 5 to 35% v/v in said emulsion.

41. The emulsion of any of claims 28-40, wherein the therapeutic agent has a concentration selected from the range of 0.1 mg mL⁻¹ to 50 mg mL⁻¹ relative to the hydrophobic liquid in said emulsion; and wherein the semi-fluorinated block copolymers have a concentration selected from the range of 5 mg mL⁻¹ to 50 mg mL⁻¹.

42. The emulsion of any of claims 1-45, wherein said aqueous solution comprises a saline solution.

43. The emulsion of any of claims 1-42, wherein said emulsions contain individual oil droplet core particles having an average diameter less than or equal to 500 nanometers.

44. The emulsion of claim 43, wherein said droplets have an average diameter less than or equal to 400 nanometers.

45. The emulsion of any of claims 42-44, wherein said droplets have a hydrophobic core comprising said hydrophobic blocks of said semi-fluorinated block copolymers.

46. The emulsion of any of claims 42-45, wherein said emulsions have a hydrophilic exterior shell comprising said hydrophilic blocks of said semi-fluorinated block copolymers.

47. The emulsion of any of claims 42-46, wherein said emulsions have a fluorophilic intermediate shell comprising said fluorophilic blocks of said semi-fluorinated block copolymers.

48. The emulsion of claim 47, wherein said therapeutic compound is noncovalently associated with said hydrophobic core.

49. The emulsion of any of claims 43-48, wherein said droplets comprise self-assembled supramolecular structures within the aqueous solution.

50. The emulsion of any of claims 1-50, wherein said emulsion is for administration to a subject in need thereof via intravenous injection.

51. The emulsion of any of claims 1-50, wherein said emulsion provides enhanced stability as compared to an emulsion in the absence of the semi-fluorinated block copolymer.

52. The emulsion of any of claims 1-50 wherein the emulsion provides an extension of the biological half-life of the therapeutic agent as compared to an emulsion in the absence of the semi-fluorinated block copolymer.

53. The emulsion of claim 52, wherein the enhanced stability is due to reduced Ostwald ripening.

54. A method of delivering an imaging agent and a therapeutic agent to a subject in need thereof, said method comprising the steps of:
(a) providing an emulsion, said emulsion comprising an oil in water emulsion comprising:
a hydrophobic liquid comprising a therapeutic agent;
an aqueous solution;
semi-fluorinated block copolymers; wherein each of said semi-fluorinated block copolymers independently comprises a hydrophilic block, a hydrophobic block and a fluorophilic block; wherein said fluorophilic block of each of said semi-fluorinated block copolymers is provided between said hydrophobic block and said hydrophilic block; and
an imaging agent comprising a fluorous compound, and
(b) administering said emulsion to said subject.

55. The method of claim 54, wherein said therapeutic agent is released from said emulsion after delivery to the subject.

56. A method of making a theranostic emulsion, said method comprising the steps of:
(a) providing
(i) a hydrophobic liquid,
(ii) am aqueous solution,
(iii) semi-fluorinated block copolymers; wherein each of said semi-fluorinated block copolymers independently comprises a hydrophilic block, a hydrophobic block and a fluorophilic block; wherein said fluorophilic block of each of said semi-fluorinated block copolymers is provided between said hydrophobic block and said hydrophilic block;
(iv) a therapeutic agent;
(v) an imaging agent comprising a fluorous compound; and
(b) emulsifying said theranostic formulation to create the emulsion.

57. The method of any of claims 54-56, wherein said emulsion provides an extension of the biological half-life of the therapeutic agent as compared to an emulsion in the absence of the semi-fluorinated block copolymer and wherein said emulsion provides enhanced stability as compared to an emulsion in the absence of the semi-fluorinated block copolymer.

58. The method any of claims 54-57, wherein each of said semi-fluorinated block copolymers independently has the formula (FX4A) or (FX4B): [chemical structure]
